SEQUENCE LISTING (12 sequences)

SEQ ID NO: 1 - DNA, 343 bp, Homo sapiens
  CDS (151)..(342)
ggacaccactggggccccagggtgtggcaagtgaggatggcaagggttttgctaaacaaatcctctgcccgctccccgccccgggctcactccatgtgaggccccagtcggggcagccacctgccgtgcctgttggaagttgcctctgccatgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggt

SEQ ID NO: 2 - DNA, 305 bp, Homo sapiens
  CDS (4)..(303)
aggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggt

SEQ ID NO: 3 - DNA, 787 bp, Homo sapiens
  CDS (3)..(785)
aggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcaggt

SEQ ID NO: 4 - DNA, 208 bp, Homo sapiens
  CDS (3)..(206)
agctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccaggt

SEQ ID NO: 5 - DNA, 125 bp, Homo sapiens
  CDS (3)..(122)
agaagcccgtgtcccggtgctcgcggcagtgccaggagggccaggtgcgccgggtcaaggggttccactcctgctgctacgactgtgtggactgcgaggcgggcagctaccggcaaaacccaggt

SEQ ID NO: 6 - DNA, 961 bp, Homo sapiens
  CDS (2)..(958)
agacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 7 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELSQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 8 - protein, 490 aa, Mus musculus
MPALAIMGLSLAAFLELGMGASLCLSQQFKAQYILGGPLGSTEEATLNQRTQPNSIPNRFSPLLFLAMKMAVEENGSAGLRYDLFTSEPVVTMKSSLMFLAKVGSQSIAAYCNYTQYQPRVLAVIGPHSSELALITGKFFSFLMQVSSASMDRSDRETFPSFFTVSDRVQLQAVVTLLQNFSNWVAALGSDDDREGLSIFSSLANARGIIAHEGLVPQHDTSGQQLGKVLDVLRQVNQSKVQVVLASARAVYSLFSYSIHHGLSPKVWVAESLTSDLVMTLPNIARVTVLGFLQRGALLPEFSHYVETHLALAADPAFASLNAELDLEEHVMGQRCPRDDIMLQNLSSGLLQNLSAGQLHHQIFATYAVYSVQAHNTLQNVSHHVSEHVLPWQLENMYNMSHARDLTLQAENVDMEYDLKMWVWQSPTPVLHTVGTFNGTQLQQSKMYWPGNQPVQSRQKDQVRRVKGFHSYDVDKAGSYRKHPDDFTTP

SEQ ID NO: 9 - protein, 480 aa, Rattus norvegicus
MLFWAAHLLLSLQLVYCWAFSCQRTESSPGFSLPFLLAGSLHGDCLQVRHRPLVTSDRPDSFNGHYHLFQRFTVEENSSANITYELYVSESANVYATLRVLALQGPRHIEIQKDLRNHSSKVVAFIPDNTDHAVTTAALLGPLMLVSEASSVVSAKRKFPSFLTVSDRHQVEVMVQLLQSFGVWISLIGSDYGQLGVQALEELAVPRGIVAFKDIVPFSARVGDPRMQSMMQHLAQARTTVVVSNRHLARVFFRSVVLANLTGKVWVAEDAISTYITSVTGIQGITVLGVAVQQRQVPGLKEFEESYVRAVTAAPSAPEGSWSTCNQLRECHTFTTRNMPTLGAFSMSAAYRVYEVAVHGHQLLGTSEISRGPVYPWQLQQIYKVNLLHENTVADNDTLGYYDIIAWDWNGPEWTFEIIGSASLSPVHDINKTKIQWHGKNNQPVVTTDLAHHRVVVGSHHFEVPEAGTFLNMSELHIQP

SEQ ID NO: 10 - protein, 484 aa, Rattus norvegicus
MGPQARTLCLLSLLLHVLPKPGKLVENSDFHLAYLLGGTLHANVKSISHLSYLQVPKNEFTMKVLYNLMQRFAVEENCSSGVLYEMVVYLSNNIHPGLYFLAQDDDLLPILKDYSQYMPHVVAVIPDNSESAITVSNILSHLIQITSAISDKRDKRHFPSMLTVSATHHIEAMVQLMVHFQNWIVVLVSDDDRENSHLLSQRLTKTSDIIAFQEVLPIPESSQVMRSEEQRQLDNILDKLRRTSARVVVSPELSLYSFFHEVLRWNFTGFVWIAESAIDPVLHNLTELRHTTFLGVTIQRVSIPGFSQFRVRRDKPGYPVPNTTNLRTTNQDDACLNTTKSFNNILILSGERVVYSVYSVAVHAHRLLGNRVRTKQKVYPWQLREIWHVNTLLGNRLFQQDMPMLLDIIQWQWDLSQNPFQSIASYSPTSKRTYINNVSWYTPNNTPVMSKSQPQMKKSVGLHPFELDMPGTYLNRSADEFNLS

SEQ ID NO: 11 - protein, 528 aa, Mus musculus
MAWFGYCLALLALTWHSSAYGPDQRAQKKIILGGPIHFGVSAKDQDLKSRPESVEIRYNFRFRWLQIFAIEESSPANMTYRIFTNTVSKALEATLSFVAQNKIDSLNLDEFCNCSEHIPSTIAVVATGSGVSTAVANLLGLYIQVSASSSRLSNKNQFKSFLTINDEHQATAMADIIEYFRNWVGTIAADDDRPGIEKFREEAEERDIIDFSELISQYSDEEEIQQVVEVIQNSTAKIVVSSGPDLEPLIKEIVRRNITGRIWLAEAASSSLIAMPEYFHVVGTIGFGLKAGQIPGFREFLQKVHPRKSVHNGFAKEFWEETFNHLQDGAKGPLPVDTFVRSHEEGGNRLLNSSTAFRPLTGDENINSVETPYMDYEHLRISYNVYLVSIHAQDIYTLPGRGLFTNGSADIKKVEAWQVKHLRHLNTNNMGEQVTECDLVGNYSIINWHLSPEDGSIVFKEVGYYNVYAKKGERFINEGKILWSGFSREPFNSRDQATRKGIIEGEPTFEAECPDGEYSGETDASADK

SEQ ID NO: 12 - protein, 500 aa, Mus musculus
FFPMIFLEMSILPRMPDRKVLLAGASSQRSVARMDVIIGASVHHQPPAEKVPERKGEIREQYIQRVEFHTLDKADPVNITSEIRSWHSSVALEQSIEFIRDSLISIRDEKDGLNRCLPDGQTLPPGRTKKPIAGVIPGSSSVAIQVQNLLQLDIQIASATSIDSDKTLYKYFLVVSDTLQARAMLDIVKRYNTYVSAVHTEGNESGMDAFKELAAQEGLIAHSDKIYSNAGEKSFDRLLRKLRERLPKARVVCCEGMTVRGLLSAMRRLGVVGEFSLIGDGADRDEVIEGYEVEANGITIKLQSPEVRSFDDYFLKLRLDTNTRNPWFPEFWQHRFQRLPGHLLENPNFKKVTGNESLEENYVQDSKMGFVINIAMHGQNMHHALPGHVGLDAMKPIDGRKLDFLIKSSVGVSGEEVWEKDAPGRYDIMNLQYTEANRYDYVHVGTWHEGVNIDDYKIQMNKSGMRVSEPLKQIKVIRKGEVSWITAKENEFVQDEFTRA

(19) United States
(12) Patent Application Publication
(10) Pub. No.: US 2004/0219632 A1
(43) Pub. Date: Nov. 4, 2004

(54) T1r3 a novel taste receptor

(76) Inventors: Robert Margolskee, Upper Montclair, NJ (US); Marianna Max, West Orange, NJ (US); Harel Weinstein, New York, NY (US); Fabien Campagne, Astoria, NY (US); Gopi Y. Shanker, New York, NY (US)

Correspondence Address: James F Haley Jr, Fish & Neave, 1251 Avenue of the Americas, New York, NY, 10020-1104, US

(21) Appl. No.: 10/475,620

(22) PCT Filed: Apr. 22, 2002
(86) PCT No.: PCT/US02/12656

Related U.S. Application Data
(60) Provisional application No. 60/285,209, filed on Apr. 20, 2001.

(51) Int. Cl. C07K 14/705; C12N 5/06

(52) U.S. Cl. 435/69.1; 435/320.1; 435/325; 530/350; 536/23.5

(57) ABSTRACT

The present invention relates to the discovery, identification and characterization of a receptor protein, referred to herein as T1R3, which is expressed in taste receptor cells and associated with the perception of bitter and sweet taste. The invention encompasses T1R3 nucleotides, host cell expression systems, T1R3 proteins, fusion protein, transgenic animals that express a T1R3 transgene, and recombinant “knock-out” animals that do not express T1R3. The invention further relates to methods for identifying modulators of the T1R3-mediated taste response and the use of such modulators to either inhibit or promote the perception of bitterness or sweetness. The modulators of T1R3 activity may be used as flavor enhancers in foods, beverages and pharmaceuticals.

### BACKGROUND

The present invention relates to the discovery, identification and characterization of a G protein coupled receptor, referred to herein as T1R3, which is expressed in taste receptor cells and associated with the perception of sweet taste. The invention encompasses T1R3 nucleotides, host cell expression systems, T1R3 proteins, fusion proteins, polypeptides and peptides, antibodies to the T1R3 protein, transgenic animals that express a T1R3 transgene, and recombinant “knock-out” animals that do not express T1R3. The invention further relates to methods for identifying modulators of the T1R3-mediated taste response and the use of such modulators to either inhibit or promote the perception of sweetness. The modulators of T1R3 activity may be used as flavor enhancers in foods, beverages and pharmaceuticals.

The sense of taste plays a critical role in the life and nutritional status of humans and other organisms. Human taste perception may be categorized according to four well-known and widely accepted descriptors, sweet, bitter, salty and sour (corresponding to particular taste qualities or modalities), and two more controversial qualities: fat and amino acid taste. The ability to identify sweet-tasting foodstuffs is particularly important as it provides vertebrates with a means to seek out needed carbohydrates with high nutritive value. The perception of bitter, on the other hand, is important for its protective value, enabling humans to avoid a plethora of potentially deadly plant alkaloids and other environmental toxins such as ergotamine, atropine and strychnine. During the past few years a number of molecular studies have identified components of bitter-responsive transduction cascades, such as α-gustducin (1, 2), Gγ13 (3) and the T2R/TRB receptors (4-6). However, the components of sweet taste transduction have not been identified so definitively (7, 8), and the elusive sweet-responsive receptors have neither been cloned nor physically characterized.

Based on biochemical and electrophysiological studies of taste cells the following two models for sweet transduction have been proposed and are widely accepted (7, 8). First, a GPCR-Gₛ-cAMP pathway—sugars are thought to bind to and activate one or more G protein coupled receptors (GPCRs) linked to Gₛ; receptor-activated Gαₛ activates adenylyl cyclase (AC) to generate cAMP; cAMP activates protein kinase A which phosphorylates a basolateral K⁺ channel, leading to closure of the channel, depolarization of the taste cell, voltage-dependent Ca⁺⁺ influx and neurotransmitter release. Second, a GPCR-G_{q}/GβΓ-IP₃ pathway—artificial sweeteners presumably bind to and activate one or more GPCRs coupled to PLCβ2 by either the α subunit of G_{q} or by Gβγ subunits; activated Gα_{q} or released Gβγ activates PLCβ2 to generate inositol trisphosphate (IP₃) and diacyl glycerol (DAG); IP₃ and DAG elicit Ca⁺⁺ release from internal stores, leading to depolarization of the taste cell and neurotransmitter release. Progress in this field has been limited by the inability to clone sweet-responsive receptors.

Genetic studies in mice have identified two loci, sac (determines behavioral and electrophysiological responsiveness to saccharin, sucrose and other sweeteners) and dpa (determines responsiveness to D-phenylalanine), that provide major contributions to differences between sweet-sensitive and sweet-insensitive strains of mice (9-12). Sac has been mapped to the distal end of mouse chromosome 4, and dpa mapped to the proximal portion of mouse chromosome 4 (13-16). The orphan taste receptor T1R1 was tentatively mapped to the distal region of chromosome 4, hence, it was proposed as a candidate for sac (17). However, detailed analysis of the recombination frequency between T1R1 and markers close to sac in F2 mice indicates that T1R1 is rather distant from sac (˜5 cM away according to genetic data of Li et al (16); and more than a million base pairs away from D18346, the marker closest to sac. Another orphan taste receptor, T1R2, also maps to mouse chromosome 4, however, it is even further away from D18346/sac than is T1R1.

To thoroughly understand the molecular mechanisms underlying taste sensation, it is important to identify each molecular component in the taste signal transduction pathways. The present invention relates to the cloning of a G protein coupled receptor, T1R3, that is believed to be involved in taste transduction and may be involved in the changes in taste cell responses associated with sweet taste perception.

### SUMMARY OF THE INVENTION

The present invention relates to the discovery, identification and characterization of a novel G protein coupled receptor referred to hereafter as T1R3, that participates in the taste signal transduction pathway. T1R3 is a receptor protein with a high degree of structural similarity to the family 3 G protein coupled receptors (herein after GPCR). As demonstrated by Northern: Blot analysis, expression of the T1R3 transcript is tightly regulated, with the highest level of gene expression found in taste tissue. In situ hybridization indicates that T1R3 is selectively expressed in taste receptor cells, but is absent from the surrounding lingual epithelium, muscle or connective tissue. Moreover, T1R3 is highly expressed in taste buds from fungiform, foliate and circumvallate papillae.

The present invention encompasses T1R3 nucleotides, host cells expressing such nucleotides and the expression products of such nucleotides. The invention encompasses T1R3 protein, T1R3 fusion proteins, antibodies to the T1R3 receptor protein and transgenic animals that express a T1R3 transgene or recombinant knock-out animals that do not express the T1R3 protein.

Further, the present invention also relates to screening methods that utilize the T1R3 gene and/or T1R3 gene products as targets for the identification of compounds which modulate, i.e., act as agonists or antagonists, of T1R3 activity and/or expression. Compounds which stimulate taste responses similar to those of sweet tastants can be used as additives to act as flavor enhancers in foods, beverages or pharmaceuticals by increasing the perception of sweet taste. Compounds which inhibit the activity of the T1R3 receptor may be used to block the perception of sweetness.

The invention is based, in part, on the discovery of a GPCR expressed at high levels in taste receptor cells. In taste transduction, sweet compounds are thought to act via a second messenger cascade utilizing PLCβ2 and IP₃. Co-localization of α-gustducin, PLCβ₂ Gβ3 and Gγ13 and T1R3 to one subset of taste receptor cells indicates that they may function in the same transduction pathway.

### DEFINITIONS

As used herein, italicizing the name of T1R3 shall indicate the T1R3 gene, T1R3 DNA, cDNA, or RNA, in contrast to its encoded protein product which is indicated by the name of T1R3 in the absence of italicizing. For example, “T1R3” shall mean the T1R3 gene, T1R3 DNA, cDNA, or RNA whereas “T1R3” shall indicate the protein product of the T1R3 gene.

### BRIEF DESCRIPTION OF THE DRAWINGS

FIG. 1A. Synteny between human 1 p36.33 and mouse 4pter chromosomal regions near the mouse Sac locus. Shaded circles indicate the approximate location of the predicted start codons for each gene; arrows indicate the full span of each gene including both introns and exons; arrowheads indicate the approximate location of each polyadenylation signal. Genes indicated by lowercase letters were predicted by Genscan and named according to their closest homolog. Genes indicated by capital letters (T1R3 and DVL1) were experimentally identified and verified. The mouse marker D18346 indicated is closely linked to the Sac locus and lies within the predicted pseudouridine synthase-like gene. The region displayed corresponds to ˜45,000 bp; the bottom scale marker indicates kilobases (K).

FIG. 1B. The nucleotide and predicted amino acid sequences of human T1R3. The ends of the introns are indicated in highlighted lower case letters.

FIG. 1C. Predicted secondary structure of human T1R3. T1R3 is predicted to have seven transmembrane helices and a large N-terminal domain. Placement of the transmembrane segments was according to the TMpred program. Placement of the dimerization and ligand binding domain, and the cysteine-rich domain were based on the mGluR1 receptor and other family 3 GPCRs (19).

FIG. 2A. Distribution of T1R3 mRNA in mouse tissues and mouse taste cells. Autoradiogram of a Northern blot hybridized with mouse T1R3 cDNA. Each lane contained 25 μg of total RNA isolated from the following mouse tissues: circumvallate and foliate papillae-enriched lingual tissue (Taste), lingual tissue devoid of taste buds (Non-Taste), brain, retina, olfactory epithelium (Olf Epi), stomach, small intestine (Small Int), thymus, heart, lung, spleen, skeletal muscle (Ske Mus), liver, kidney, uterus and testis. A 7.2 kb transcript was detected only in the taste tissue, and a slightly larger transcript was detected in testis. The blot was exposed to X-ray film for three days. The same blot was stripped and reprobed with a β-actin cDNA (lower panel) and exposed for one day. The size of the RNA marker (in kilobases) is indicated in the right margin.

FIG. 2B. The genomic sequence of the Sac region from mouse was used as a query to search the mouse expressed sequence tag (est) database. Matches to the est database are shown in solid red and indicate exons; gaps in a particular est match are shown by black hashed lines and indicate an intron. The clustered nature of the est matches demarcates the extent of each of the genes within this region. The near absence of ests at the position of T1R3 is consistent with the highly restricted pattern of expression seen in FIG. 2a.

FIG. 3A. T1R3 expression in taste receptor cells. Photomicrographs of frozen sections of mouse taste papillae hybridized with ³³P-labelled antisense RNA probes for T1R3 and α-gustducin. Bright-field images of circumvallate (a), foliate (b), and fungiform (c) papillae hybridized to the antisense T1R3 probe demonstrate taste bud-specific expression of T1R3. Control bright-field images of circumvallate (e), foliate (f), and fungiform papillae (g) hybridized to the sense T1R3 probe showed no nonspecific binding. The level of expression and broad distribution of T1R3 expression in taste buds was comparable to that of α-gustducin as shown in the bright field image of circumvallate papilla hybridized to antisense α-gustducin probe (d). The control bright field image of circumvallate papilla hybridized to the sense α-gustducin probe (h) showed no nonspecific binding.

FIG. 3B. Profiling the pattern of expression of T1R3, α-gustducin, Gγ13 and PLCβ2 in taste tissue and taste cells. Left panel: Southern hybridization to RT-PCR products from murine taste tissue (T) and control non-taste lingual tissue (N). 3′-region probes from T1R3, α-gustducin (Gust), Gγ13, PLCβ2 and glyceraldehyde 3-phosphate dehydrogenase (G3PDH) were used to probe the blots. Note that T1R3, α-gustducin, Gγ13 and PLCβ2 were all expressed in taste tissue, but not in non-taste tissue. Right panel: Southern hybridization to RT-PCR products from 24 individually amplified taste receptor cells. 19 cells were GFP-positive (+), 5 cells were GFP-negative (−). Expression of α-gustducin, Gγ13 and PLCβ2 was fully coincident. Expression of T1R3 overlapped partially with that of α-gustducin, Gγ13 and PLCβ2. G3PDH served as a positive control to demonstrate successful amplification of products.

FIG. 4. Co-localization of T1R3 PLCβ2 and α-gustducin in taste receptor cells of human circumvallate papillae. (a, c) Longitudinal sections from human circumvallate papillae were labeled with rabbit antisera directed against a C-terminal peptide of human T1R3, along with a Cy3-conjugated anti-rabbit secondary antibody. (b) T1R3 immunoreactivity in longitudinal sections from human papillae was blocked by pre-incubation of the T1R3 antibody with the cognate peptide. (d) A longitutidinal section adjacent to that in sections of human fungiform papillae double immunostained for T1R3 (h) and α-gustducin (i). The overlay of the two images is shown in (j). Magnification was 200X (a-d) or 400X (e-j).

FIG. 5A. mT1R3 allelic differences. mT1R3 allelic differences between eight inbred mouse strains. All non-taster strains showed identical sequences and were grouped in one row. In the bottom row the amino acid immediately before the position number is always from the non-tasters, while the amino acid immediately before the position number is from whichever tasters differed at that position from the non-tasters. The two columns in bold represent positions where all tasters differed from non-tasters and where the differences in nucleotide sequence result in amino acid substitutions. Nucleotide differences that do not alter the encoded amino acid are indicated as s: silent. Nucleotide differences within introns are indicated as i: intron.

FIG. 5B. Genealogy of the inbred strains of mice analyzed in (a). The year in which the strains were developed is indicated between brackets following the stain name. The laboratories in which these mice were established are indicated.

FIG. 6. The amino acid sequence of mouse T1R3 is aligned with that of two other rat taste receptors (rT1R1 and rT1R2), the murine extracellular calcium sensing (mECaSR) and the metabotropic glutamate type 1 (mGluR1) receptors. Regions of identity among all five receptors are indicated by white letters on black; regions where one or more of these receptors share identity with T1R3 are indicated by black letters on gray. Boxes with dashed lines indicate regions predicted to be involved in dimerization (based upon the solved structure for the amino terminal domain of mGluR1); filled circles indicate predicted ligand binding residues based on mGluR1; blue lines linking cysteine residues indicate predicted intermolecular disulfide bridges based on mGluR1. Amino acid sequences noted above the alignment indicate polymorphisms that are found in all strains of nontaster mice. The predicted N-linked glycosylation site conserved in all five receptors is indicated by a black squiggle; the predicted N-linked glycosylation site specific to T1R3 in nontaster strains of mice is indicated by the red squiggle.

FIG. 7. The predicted three dimensional structure of the amino-terminal domain (ATD) of T1R3 modeled on that of mGluR1 (19) using the Modeller program. The model shows a homodimer of T1R3. (a) The view from the “top” of the dimer looking down from the extracellular space toward the membrane. (b) The T1R3 dimer viewed from the side. In this view the transmembrane region (not displayed) would attach to the bottom of the dimer. (c) The T1R3 dimer is viewed from the side as in (b), except the two dimers have been spread apart (indicated by the double headed arrow) to reveal the contact surface. A space-filling representation (colored red) of three glycosyl moieties (N-acetyl-galactose-N-acetyl-galactose-Mannose) has been added at the novel predicted site of glycosylation of non-taster mT1R3. Note that the addition of even three sugar moieties at this site is sterically incompatible with dimerization. Regions of T1R3 corresponding to those of mGluR1 involved in dimerization are shown by space filling amino acids. The four different segments that form the predicted dimerization surface are color-coded in the same way as are the dashed boxes in FIG. 5. The portions of the two molecules outside of the dimerization region are represented by a backbone tracing. The two polymorphic amino acid residues of T1R3 that differ in taster vs. non-taster strains of mice are within the predicted dimerization interface nearest the amino terminus (colored light blue). The additional N-glycosylation site at aa58 unique to the non-taster form of T1R3 is indicated in each panel by the straight arrows.

### DETAILED DESCRIPTION OF THE INVENTION

T1R3 is a novel receptor that participates in receptor-mediated taste signal transduction and belongs to the family 3 G protein coupled receptors. The present invention encompasses T1R3 nucleotides, T1R3 proteins and peptides, as well as antibodies to the T1R3 protein. The invention also relates to host cells and animals genetically engineered to express the T1R3 receptor or to inhibit or “knock-out” expression of the animal's endogenous T1R3.

The invention further provides screening assays. designed for the identification of modulators, such as agonists and antagonists, of T1R3 activity. The use of host cells that naturally express T1R3 or genetically engineered host cells and/or animals offers an advantage in that such systems allow the identification of compounds that affect the signal transduced by the T1R3 receptor protein.

Various aspects of the invention are described in greater detail in the subsections below.

### The T1R3 Gene

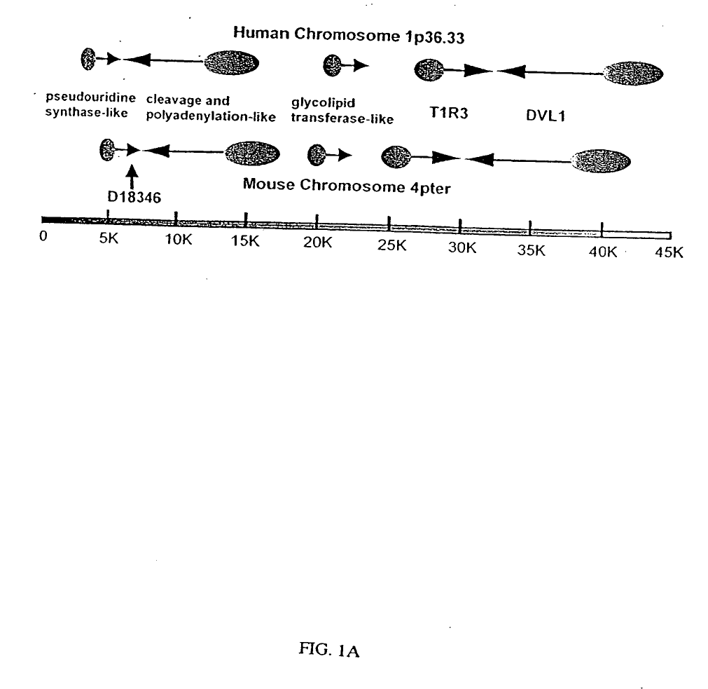

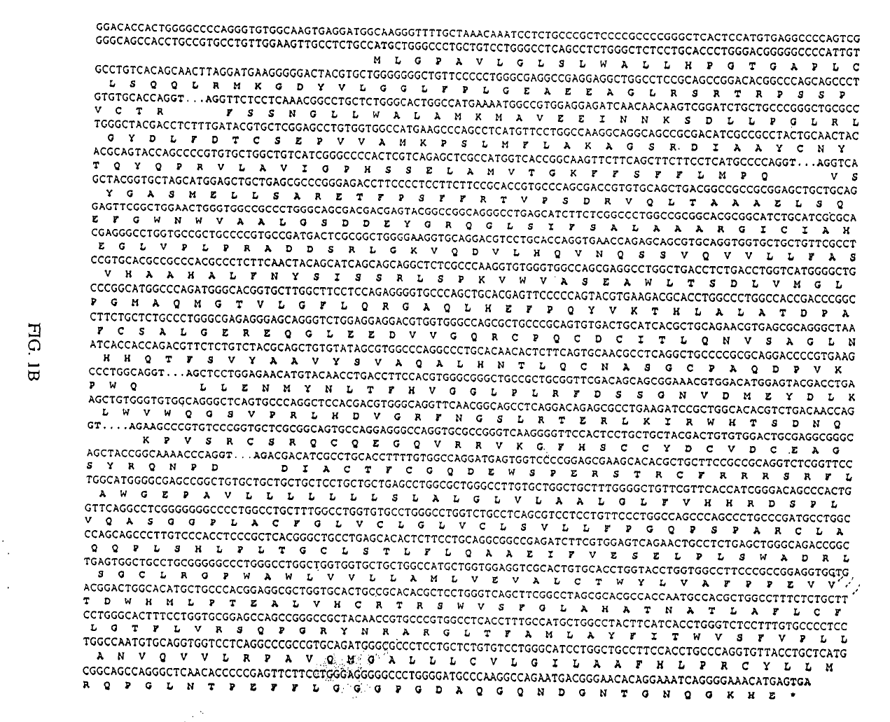

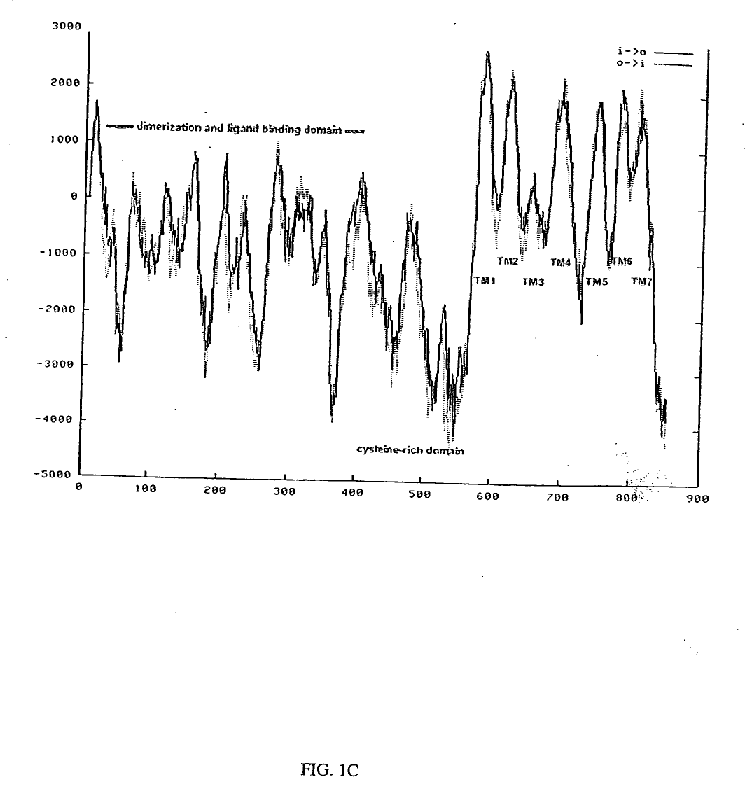

The cDNA sequence and deduced amino acid sequence of human T1R3 is shown in FIG. 1B. The T1R3 nucleotide sequences of the invention include: (a) the DNA sequence shown in FIG. 1B; (b) nucleotide sequences that encode the amino acid sequence shown in FIG. 1B; (c) any nucleotide sequence that (i) hybridizes to the nucleotide sequence set forth in (a) or (b) under stringent conditions, e.g., hybridization to filter-bound DNA in 0.5 M NaHPO₄, 7% sodium dodecyl sulfate (SDS), 1 mM EDTA at 65EC, and washing in 0.1×SSC/0.1% SDS at 68EC (Ausubel F. M. et al., eds., 1989, Current Protocols in Molecular Biology, Vol. I, Green Publishing Associates, Inc., and John Wiley & sons, Inc., New York, at p. 2.10.3) and (ii) encodes a functionally equivalent gene product; and (d) any nucleotide sequence that hybridizes to a DNA sequence that encodes the amino acid sequence shown in FIG. 1B, under less stringent conditions, such as moderately stringent conditions, e.g., washing in 0.2×SSC/0.1% SDS at 42EC (Ausubel et al., 1989 supra), yet which still encodes a functionally equivalent T1R3 gene product. Functional equivalents of the T1R3 protein include naturally occurring T1R3 present in species other than humans. The invention also includes degenerate variants of sequences (a) through (d). The invention also includes nucleic acid molecules, that may encode or act as T1R3 antisense molecules, useful, for example, in T1R3 gene regulation (for and/or as antisense primers in amplification reactions of T1R3 gene nucleic acid sequences).

In addition to the T1R3 nucleotide sequences described above, homologs of the T1R3 gene present in other species can be identified and readily isolated, without undue experimentation, by molecular biological techniques well known in the art. For example, cDNA libraries, or genomic DNA libraries derived from the organism of interest can be screened by hybridization using the nucleotides. described herein as hybridization or amplification probes.

The invention also encompasses nucleotide sequences that encode mutant T1R3s, peptide fragments of the T1R3, truncated T1R3, and T1R3 fusion proteins. These include, but are not limited to nucleotide sequences encoding polypeptides or peptides corresponding to functional domains of T1R3, including but not limited to, the ATD (amino terminal domain) that is believed to be involved in ligand binding and dimerization, the cysteine rich domain, and/or the transmembrane spanning domains of T1R3, or portions of these domains; truncated T1R3s in which one or two domains of T1R3 is deleted, e.g., a functional T1R3 lacking all or a portion of the ATD region. Nucleotides encoding fusion proteins may include but are not limited to full length T1R3, truncated T1R3 or peptide fragments of T1R3 fused to an unrelated protein or peptide such as an enzyme, fluorescent protein, luminescent protein, etc., which can be used as a marker.

Based on the model of T1R3's structure, it is predicted that T1R3 dimerizes to form a functional receptor. Thus, certain of these truncated or mutant T1R3 proteins may act as dominant-negative inhibitors of the native T1R3 protein. T1R3 nucleotide sequences may be isolated using a variety of different methods known to those skilled in the art. For example, a cDNA library constructed using RNA from a tissue known to express T1R3 can be screened using a labeled T1R3 probe. Alternatively, a genomic library may be screened to derive nucleic acid molecules encoding the T1R3 receptor protein. Further, T1R3 nucleic acid sequences may be derived by performing PCR using two oligonucleotide primers designed on the basis of the T1R3 nucleotide sequences disclosed herein. The template for the reaction may be cDNA obtained by reverse transcription of mRNA prepared from cell lines or tissue known to express T1R3.

The invention also encompasses (a) DNA vectors that contain any of the foregoing T1R3 sequences and/or their complements (i.e., antisense); (b) DNA expression vectors that contain any of the foregoing T1R3 sequences operatively associated with a regulatory element that directs the expression of the T1R3 coding sequences; (c) genetically engineered host cells that contain any of the foregoing T1R3 sequences operatively associated with a regulatory element that directs the expression of the T1R3 coding sequences in the host cell; and (d) transgenic mice or other organisms that contain any of the foregoing T1R3 sequences. As used herein, regulatory elements include but are not limited to inducible and non-inducible promoters, enhancers, operators and other elements known to those skilled in the art that drive and regulate expression.

### T1R3 Proteins and Polypeptides

T1R3 protein, polypeptides and peptide fragments, mutated, truncated or deleted forms of the T1R3 and/or T1R3 fusion proteins can be prepared for a variety of uses, including but not limited to the generation of antibodies, the identification of other cellular gene products involved in the regulation of T1R3 mediated taste transduction, and the screening for compounds that can be used to modulate taste perception such as novel sweetners and taste modifiers.

FIG. 1B shows the deduced amino acid sequence of the human T1R3 protein. The T1R3 amino acid sequences of the invention include the amino acid sequence shown in FIG. 1B. Further, T1R3s of other species are encompassed by the invention. In fact, any T1R3 protein encoded by the T1R3 nucleotide sequences described in Section 5.1, above, is within the scope of the invention.

The invention also encompasses proteins that are functionally equivalent to the T1R3 encoded by the nucleotide sequences described in Section 5.1, as judged by any of a number of criteria, including but not limited to the ability of a sweet tastant to activate T1R3 in a taste receptor cell, leading to transmitter release from the taste receptor cell into the synapse and activation of an afferent nerve. Such functionally equivalent T1R3 proteins include but are not limited to proteins having additions or substitutions of amino acid residues within the amino acid sequence encoded by the T1R3 nucleotide sequences described, above, in Section 5.1, but which result in a silent change, thus producing a functionally equivalent gene product.

Peptides corresponding to one or more domains of T1R3 (e.g., amino terminal domain, the cysteine rich domain and/or the transmembrane spanning domains), truncated or deleted T1R3s (e.g., T1R3 in which the amino terminal domain, the cysteine rich domain and/or the transmembrane spanning domains is deleted) as well as fusion proteins in which the full length T1R3, a T1R3 peptide or a truncated T1R3 is fused to an unrelated protein are also within the scope of the invention and can be designed on the basis of the T1R3 nucleotide and T1R3 amino acid sequences disclosed herein. Such fusion proteins include fusions to an enzyme, fluorescent protein, or luminescent protein which provide a marker function.

While the T1R3 polypeptides and peptides can be chemically synthesized (e.g., see Creighton, 1983, Proteins: Structures and Molecular Principles, W. H. Freeman & Co., N.Y.), large polypeptides derived from T1R3 and the full length T1R3 itself may be advantageously produced by recombinant DNA technology using techniques well known in the art for expressing a nucleic acid containing T1R3 gene sequences and/or coding sequences. Such methods can be used to construct expression vectors containing the T1R3 nucleotide sequences described in Section 5.1 and appropriate transcriptional and translational control signals. These methods include, for example, in vitro recombinant DNA techniques, synthetic techniques, and in vivo genetic recombination. (See, for example, the techniques described in Sambrook et al., 1989, supra, and Ausubel et al., 1989, supra).

A variety of host-expression vector systems may be util ized to express the T1R3 nucleotide sequences of the invention. Where the T1R3 peptide or polypeptide is expressed as a soluble derivative (e.g., peptides corresponding to the amino terminal domain the cysteine rich domain and/or the transmembrane spanning domain) and is not secreted, the peptide or polypeptide can be recovered from the host cell. Alternatively, where the T1R3 peptide or polypeptide is secreted the peptide or polypeptides may be recovered from the culture media. However, the expression systems also include engineered host cells that express T1R3 or functional equivalents, anchored in the cell membrane. Purification or enrichment of the T1R3 from such expression systems can be accomplished using appropriate detergents and lipid micelles and methods well known to those skilled in the art. Such engineered host cells themselves may be used in situations where it is important not only to retain the structural and functional characteristics of the T1R3, but to assess biological activity, i.e., in drug screening assays.

The expression systems that may be used for purposes of the invention include but are not limited to microorganisms such as bacteria transformed with recombinant bacteriophage, plasmid or cosmid DNA expression vectors containing T1R3 nucleotide sequences; yeast transformed with recombinant yeast expression vectors containing T1R3 nucleotide sequences or mammalian cell systems harboring recombinant expression constructs containing promoters derived from the genome of mammalian cells or from mammalian viruses.

Appropriate expression systems can be chosen to ensure that the correct modification, processing and sub-cellular localization of the T1R3 protein occurs. To this end, eukaryotic host cells which possess the ability to properly modify and process the T1R3 protein are preferred. For long-term, high yield production of recombinant T1R3 protein, such as that desired for development of cell lines for screening purposes, stable expression is preferred. Rather than using expression vectors which contain origins of replication, host cells can be transformed with DNA controlled by appropriate expression control elements and a selectable marker gene, i.e., tk, hqprt, dhfr, neo, and hygro gene, to name a few. Following the introduction of the foreign DNA, engineered cells may be allowed to grow for 1-2 days in enriched media, and then switched to a selective media. Such engineered cell lines may be particularly useful in screening and evaluation of compounds that modulate the endogenous activity of the T1R3 gene product.

### Transgenic Animals

The T1R3 gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate T1R3 transgenic animals.

Any technique known in the art may be used to introduce the T1R3 transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci. USA 82:6148-6152); gene targeting in embryonic stem cells (Thompson et al., 1989, Cell, 56:313-321); electroporation of embryos (Lo, 1983, Mol Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229, which is incorporated by reference herein in its entirety.

The present invention provides for transgenic animals that carry the T1R3 transgene in all their cells, as well as animals which carry the transgene in some, but not all their cells, i.e., mosaic animals. The transgene may also be selectively introduced into and activated in a particular cell type by following, for example, the teaching of Lasko et al., (Lasko, M. et al., 1992, Proc. Natl. Acad. Sci. USA 89:6232-6236). The regulatory sequences required for such a cell-type specific activation will depend upon the particular cell type of interest, and will be apparent to those of skill in the art. When it is desired that the T1R3 transgene be integrated into the chromosomal site of the endogenous T1R3 gene, gene targeting is preferred. Briefly, when such a technique is to be utilized, vectors containing some nucleotide sequences homologous to the endogenous T1R3 gene are designed for the purpose of integrating, via homologous recombination with chromosomal sequences, into and disrupting the function of the nucleotide sequence of the endogenous T1R3 gene.

Once transgenic animals have been generated, the expression of the recombinant T1R3 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of T1R3 gene-expressing tissue may also be evaluated immunocytochemically using antibodies specific for the T1R3 transgene product.

### Antibodies to T1R3 Proteins

Antibodies that specifically recognize one or more epitopes of T1R3, or epitopes of conserved variants of T1R3, or peptide fragments of T1R3 are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in conjunction with compound screening schemes, as described, below, in Section 5.5, for the evaluation of the effect of test compounds on expression and/or activity of the T1R3 gene product.

For production of antibodies, various host animals may be immunized by injection with a T1R3 protein, or T1R3 peptide. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacille Calmette-Guerin) and Corynebacterium parvum.

Polyclonal antibodies comprising heterogeneous populations of antibody molecules, may be derived from the sera of the immunized animals. Monoclonal antibodies may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgS, IgA, IgD and any subclasses thereof. The hybridoma producing the mAb of this invention may be cultivated in vitro or in vivo. Production of high titres of Mabs in vivo makes this the presently preferred method of production.

In addition, techniques developed for the production of “chimeric antibodies” by splicing the genes from a mouse antibody molecule of appropriate antigen specificity together with genes from a human antibody molecule of appropriate biological activity can be used (Morrison et al., 1984, Proc. Nat'l. Acad. Sci., 81:6851-6855; Neuberger et al., 1984, Nature, 312: 604-608; Takeda et al. 1985, Nature 314: 452-454). Alternatively, techniques developed for the production of humanized antibodies (U.S. Pat. No. 5,585,089) or single chain antibodies (U.S. Pat. No. 4,946,778 Bird, 1988, Science 242: 423-426; Huston et al., 1988, Proc. Nat'l. Acad. Sci USA, 85: 5879-5883; and Ward et al., 1989, Nature 334: 544-546) may be used to produce antibodies that specifically recognize one or more epitopes of T1R3.

### Screening Assays for Drugs and Other Chemical Compounds Useful in Regulation of Taste Perception

The present invention relates to screening assay systems designed to identify compounds or compositions that modulate T1R3 activity or T1R3 gene expression, and thus, may be useful for modulation of sweet taste perception.

In accordance with the invention, a cell-based assay system can be used to screen for compounds that modulate the activity of the T1R3 and thereby, modulate the perception of sweetness. To this end, cells that endogenously express T1R3 can be used to screen for compounds. Alternatively, cell lines, such as 293 cells, COS cells, CHO cells, fibroblasts, and the like, genetically engineered to express T1R3 can be used for screening purposes. Preferably, host cells genetically engineered to express a functional T1R3 are those that respond to activation by sweet tastants, such as taste receptor cells. Further, ooyctes or liposomes engineered to express T1R3 may be used in assays developed to identify modulators of T1R3 activity.

The present invention provides for methods for identifying a compound that induces the perception of a sweet taste (a “sweetness activator”) comprising (i) contacting a cell expressing the T1R3 receptor with a test compound and measuring the level of T1R3 activation; (ii) in a separate experiment, contacting a cell expressing the T1R3 receptor protein with a vehicle control and measuring the level of T1R3 activation where the conditions are essentially the same as in part (i), and then (iii) comparing the level of activation of T1R3 measured in part (i) with the level of activation of T1R3 in part (ii), wherein an increased level of activated T1R3 in the presence of the test compound indicates that the test compound is a T1R3 activator.

The present invention also provides for methods for identifying a compound that inhibits the perception of a sweet taste (a “sweetness inhibitor”) comprising (i) contacting a cell expressing the T1R3 receptor protein with a test compound in the presence of a sweet tastant and measuring the level of T1R3 activation; (ii) in a separate experiment, contacting a cell expressing the T1R3 receptor protein with a sweet tastant and measuring the level of T1R3 activation, where the conditions are essentially the same as in part (i) and then (iii) comparing the level of activation of T1R3 measured in part (i) with the level of activation of T1R3 in part (ii), wherein a decrease level of activation of T1R3 in the presence of the test compound indicates that the test compound is a T1R3 inhibitor.

A “sweet tastant”, as defined herein, is a compound or molecular complex that induces, in a subject, the perception of a sweet taste. In particular, a sweet tastant is one which results in the activation of the T1R3 protein resulting in one or more of the following:. (i) an influx of Ca⁺² into the cell; (ii) release of Ca⁺² from internal stores; (iii) activation of coupled G proteins such as Gs and/or gustducin; (iv) activation of secon messenger-regulating enzymes such as adenylyl cyclase and/or phospholipase C. Examples of sweet tastants include but are not limited to saccharin or sucrose, or other sweetners.

In utilizing such cell systems, the cells expressing the T1R3 receptor are exposed to a test compound or to vehicle controls (e.g., placebos). After exposure, the cells can be assayed to measure the expression and/or activity of components of the signal transduction pathway of T1R3, or the activity of the signal transduction pathway itself can be assayed.

The ability of a test molecule to modulate the activity of T1R3 may be measured using standard biochemical and physiological techniques. Responses such as activation or suppression of catalytic activity, phosphorylation or dephosphorylation of T1R3 and/or other proteins, activation or modulation of second messenger production, changes in cellular ion levels, association, dissociation or translocation of signaling molecules, or transcription or translation of specific genes may be monitored. In non-limiting embodiments of the invention, changes in intracellular Ca²⁺ levels may be monitored by the fluorescence of indicator dyes such as indo, fura, etc. Additionally, changes in cAMP, cGMP, IP₃, and DAG levels may be assayed. In yet another embodiment, activation of adenylyl cyclase, guanylyl cyclase, protein kinase A and Ca²⁺ sensitive release of neurotransmitters may be measured to identify compounds that modulate T1R3 signal transduction. Further, changes in membrane potential resulting from modulation of the T1R3 channel protein can be measured using a voltage clamp or patch recording methods. In yet another embodiment of the invention, a microphysiometer can be used to monitor cellular activity.

For example, after exposure to a test compound, cell lysates can be assayed for increased intracellular levels of Ca²⁺ and activation of calcium dependent downstream messengers such as adenylyl cyclase, protein kinase A or cAMP. The ability of a test compound to increase intracellular levels of Ca²⁺, activate protein kinase A or increase cAMP levels compared to those levels seen with cells treated with a vehicle control, indicates that the test compound acts as an agonist (i.e., is a T1R3 activator) and induces signal transduction mediated by the T1R3 expressed by the host cell. The ability of a test compound to inhibit sweet tastant induced calcium influx, inhibit protein kinase A or decrease cAMP levels compared to those levels seen with a vehicle control indicates that the test compound acts as an antagonist (i.e., is a T1R3 inhibitor) and inhibits signal transduction mediated by T1R3.

In a specific embodiment of the invention, levels of cAMP can be measured using constructs containing the cAMP responsive element linked to any of a variety of different reporter genes. Such reporter genes may include but are not limited to chloramphenicol acetyltransferase (CAT), luciferase, β-glucuronidase (GUS), growth hormone, or placental alkaline phosphatase (SEAP). Such constructs are introduced into cells expressing T1R3 thereby providing a recombinant cell useful for screening assays designed to identify modulators of T1R3 activity.

Following exposure of the cells to the test compound, the level of reporter gene expression may be quantitated to determine the test compound's ability to regulate T1R3 activity. Alkaline phosphatase assays are particularly useful in the practice of the invention as the enzyme is secreted from the cell. Therefore, tissue culture supernatant may be assayed for secreted alkaline phosphatase. In addition, alkaline phosphatase activity may be measured by calorimetric, bioluminescent or chemilumenscent assays such as those described in Bronstein, I. et al. (1994, Biotechnicues 17: 172-177). Such assays provide a simple, sensitive easily automatable detection system for pharmaceutical screening.

Additionally, to determine intracellular cAMP concentrations, a scintillation proximity assay (SPA) may be utilized (SPA kit is provided by Amersham Life Sciences, Illinois). The assay utilizes ¹²⁵I-label cAMP, an anti-cAMP antibody, and a scintillant-incorporated microsphere coated with a secondary antibody. When brought into close proximity to the microsphere through the labeled cAMP-antibody complex, ¹²⁵I will excite the scintillant to emit light. Unlabeled cAMP extracted from cells competes with the ²⁵I-labeled cAMP for binding to the antibody and thereby diminishes scintillation. The assay may be performed in 96-well plates to enable high-throughput screening and 96 well-based scintillation counting instruments such as those manufactured by Wallac or Packard may be used for readout.

In yet another embodiment of the invention, levels of intracellular Ca²⁺ can be monitored using Ca²⁺ indication dyes, such as Fluo-3 and Fura-Red using methods such as those described in Komuro and Rakic, 1998, In: The Neuron in Tissue Culture. L. W. Haymes, Ed. Wiley, New York.

Test activators which activate the activity of T1R3, identified by any of the above methods, may be subjected to further testing to confirm their ability to induce a sweetness perception. Test inhibitors which inhibit the activation of T1R3 by sweet tastants, identified by any of the above methods, may then be subjected to further testing to confirm their inhibitory activity. The ability of the test compound to modulate the activity of the T1R3 receptor may be evaluated by behavioral, physiologic, or in vitro methods.

For example, a behavioral study may be performed where a test animal may be offered the choice of consuming a composition comprising the putative T1R3 activator and the same composition without the added compound. A preference for the composition comprising a test compound, indicated, for example, by greater consumption, would have a positive correlation with activation of T1R3 activity. Additionally, lack of preference by a test animal of food containing a putative inhibitor of T1R3 in the presence of a sweetner would have a positive correlation with the identification of an sweetness inhibitor.

In addition to cell based assays, non-cell based assay systems may be used to identify compounds that interact with, e.g., bind to T1R3. Such compounds may act as antagonists or agonists of T1R3 activity and may be used to regulate sweet taste perception.

To this end, soluble T1R3 may be recombinantly expressed and utilized in non-cell based assays to identify compounds that bind to T1R3. The recombinantly expressed T1R3 polypeptides or fusion proteins containing one or more of the domains of T1R3 prepared as described in Section 5.2, infra, can be used in the non-cell based screening assays. For example, peptides corresponding to the amino terminal domain that is believed to be involved in ligand binding and dimerization, the cysteine rich domain and/or the transmembrane spanning domains of T1R3, or fusion proteins containing one or more of the domains of T1R3 can be used in non-cell based assay systems to identify compounds that bind to a portion of the T1R3; such compounds may be useful to modulate the signal transduction pathway of the T1R3. In non-cell based assays the recombinantly expressed T1R3 may be attached to a solid substrate such as a test tube, microtitre well or a column, by means well known to those in the art (see Ausubel et al., supra) . The test compounds are then assayed for their ability to bind to the T1R3.

The T1R3 protein may be one which has been fully or partially isolated from other molecules, or which may be present as part of a crude or semi-purified extract. As a non-limiting example, the T1R3 protein may be present in a preparation of taste receptor cell membranes. In particular embodiments of the invention, such taste receptor cell membranes may be prepared as set forth in Ming, D. et al., 1998, Proc. Natl. Sci. U.S.A. 95:8933-8938, incorporated by reference herein. Specifically, bovine circumvallate papillae (“taste tissue”, containing taste receptor cells), may be hand dissected, frozen in liquid nitrogen, and stored at −80EC prior to use. The collected tissues may then be homogenized with a Polytron homogenizer (three cycles of 20 seconds each at 25,000 RPM) in a buffer containing 10 mM Tris at pH 7.5, 10% vol/vol glycerol, 1 mM EDTA, 1 mM DTT, 10 μg/μl pepstatin A, 10 μg/μl leupeptin, 10 μg/μl aprotinin, and 100 μM 4-(2-amino ethyl) benzenesulfoyl fluoride hydrochloride. After particulate removal by centrifugation at 1,500×g for 10 minutes, taste membranes may be collected by centrifugation at 45,000×g for 60 minutes. The pelleted membranes may then be rinsed twice, re-suspended in homogenization buffer lacking protease inhibitors, and further homogenized by 20 passages through a 25 gauge needle. Aliquots may then be either flash frozen or stored on ice until use. As another non-limiting example, the taste receptor may be derived from recombinant clones (see Hoon, M. R. et al., 1999 Cell 96, 541-551).

Assays may also be designed to screen for compounds that regulate T1R3 expression at either the transcriptional or translational level. In one embodiment, DNA encoding a reporter molecule can be linked to a regulatory element of the T1R3 gene and used in appropriate intact cells, cell extracts or lysates to identify compounds that modulate T1R3 gene expression. Appropriate cells or cell extracts are prepared from any cell type that normally expresses the T1R3 gene, thereby ensuring that the cell extracts contain the transcription factors required for in vitro or in vivo transcription. The screen can be used to identify compounds that modulate the expression of the reporter construct. In such screens, the level of reporter gene expression is determined in the presence of the test compound and compared to the level of expression in the absence of the test compound.

To identify compounds that regulate T1R3 translation, cells or in vitro cell lysates containing T1R3 transcripts may be tested for modulation of T1R3 mRNA translation. To assay for inhibitors of T1R3 translation, test compounds are assayed for their ability to modulate the translation of T1R3 mRNA in in vitro translation extracts.

In addition, compounds that regulate T1R3 activity may be identified using animal models. Behavioral, physiological, or biochemical methods may be used to determine whether T1R3 activation has occurred. Behavioral and physiological methods may be practiced in vivo. As an example of a behavioral measurement, the tendency of a test animal to voluntarily ingest a composition, in the presence or absence of test activator, may be measured. If the test activator induces T1R3 activity in the animal, the animal may be expected to experience a sweet taste, which would encourage it to ingest more of the composition. If the animal is given a choice of whether to consume a composition containing a sweet tastant only (which activates T1R3) or a composition containing a test inhibitor together with a sweet tastant, it would be expected to prefer to consume the composition containing sweet tastant only. Thus, the relative preference demonstrated by the animal inversely correlates with the activation of the T1R3 receptor.

Physiological methods include nerve response studies, which may be performed using a nerve operably joined to a taste receptor cell containing tissue, in vivo or in vitro. Since exposure to sweet tastant which results in T1R3 activation may result in an action potential in taste receptor cells that is then propagated through a peripheral nerve, measuring a nerve response to a sweet tastant is, inter alia, an indirect measurement of T1R3 activation. An example of nerve response studies performed using the glossopharyngeal nerve are described in Ninomiya, Y., et al., 1997, Am. J. Physiol. (London) 272:R1002-R1006.

The assays described above can identify compounds which modulate T1R3 activity. For example, compounds that affect T1R3 activity include but are not limited to compounds that bind to the T1R3, and either activate signal transduction (agonists) or block activation (antagonists). Compounds that affect T1R3 gene activity (by affecting T1R3 gene expression, including molecules, e.g., proteins or small organic molecules, that affect transcription or interfere with spacing events so that expression of the full length or the truncated form of the T1R3 can be modulated) can also be identified using the screens of the invention. However, it should be noted that the assays described can also identify compounds that modulate T1R3 signal transduction (e.g., compounds which affect downstream signaling events, such as inhibitors or enhancers of G protein activities which participate in transducing the signal activated by tastants binding to their receptor). The identification and use of such compounds which affect signaling events downstream of T1R3 and thus modulate effects of T1R3 on the perception of taste are within the scope of the invention.

The compounds which may be screened in accordance with the invention include, but are not limited to, small organic or inorganic compounds, peptides, antibodies and fragments thereof, and other organic compounds (e.g., peptidomimetics) that bind to T1R3 and either mimic the activity triggered by the natural tastant ligand (i.e., agonists) or inhibit the activity triggered by the natural ligand (i.e., antagonists). Such compounds may be naturally occurring compounds such as those present in fermentation broths, cheeses, plants, and fungi, for example.

Compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86); and combinatorial chemistry—derived molecular library made of D- and/or L-configuration amino acids, phosphopeptides (including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope binding fragments thereof), and small organic or inorganic molecules.

Other compounds which may be screened in accordance with the invention include but are not limited to small organic molecules that affect the expression of the T1R3 gene or some other gene involved in the T1R3 signal transduction pathway (e.g., by interacting with the regulatory region or transcription factors involved in gene expression); or such compounds that affect the activity of the T1R3 or the activity of some other intracellular factor involved in the T1R3 signal transduction pathway, such as, for example, a T1R3 associated G-protein.

### Compositions Containing Modulators of T1R3 and Their Uses

The present invention provides for methods of inducing a sweet taste resulting from contacting a taste tissue of a subject with a sweet tastant, comprising administering to the subject an effective amount of a T1R3 activator, such as a T1R3 activator identified by measuring T1R3 activation as set forth in Section 5.5 supra. The present invention also provides for methods of inhibiting the sweet taste of a composition, comprising incorporating, in the composition, an effective amount of a T1R3 inhibitor. An “effective amount” of the T1R3 inhibitor is an amount that subjectively decreases the perception of sweet taste and/or that is associated with a detectable decrease in T1R3 activation as measured by one of the above assays.

The present invention further provides for a method of producing the perception of a sweet taste by a subject, comprising administering, to the subject, a composition comprising a compound that activates T1R3 activity such as a sweetness activator identified as set forth in Section 5.5 supra. The composition may comprise an amount of activator that is effective in producing a taste recognized as sweet by a subject.

Accordingly, the present invention provides for compositions comprising sweetness activators and sweetness inhibitors. Such compositions include any substances which may come in contact with taste tissue of a subject, including but not limited to foods, beverages, pharmaceuticals, dental products, cosmetics, and wetable glues used for envelopes and stamps.

In one set of embodiments of the invention, T1R3 activators are utilized as food or beverage sweetners. In such instances, the T1R3 activators of the invention are incorporated into foods or beverages, thereby enhancing the sweet flavor of the food or beverage without increasing the carbohydrate content of the food.

In another embodiment of the invention, a sweetness activator is used to counteract the perception of bitterness associated with a co-present bitter tastant. In these embodiments, a composition of the invention comprises a bitter tastant and a sweetness activator, where the sweetness activator is present at a concentration which inhibits bitter taste perception. For example, when the concentration of bitter tastant in the composition and the concentration of sweetness activator in the composition are subjected to an assay as disclosed in Section 5.1 supra.

The present invention may be used to improve the taste of foods by increasing the perception of sweetness or by decreasing or eliminating the aversive effects of bitter tastants. If a bitter tastant is a food preservative, the T1R3 activators of the invention may permit or facilitate its incorporation into foods, thereby improving food safety. For foods administered as nutritional supplements, the incorporation of T1R3 activators of the invention may encourage ingestion, thereby enhancing the effectiveness of these compositions in providing nutrition or calories to a subject.

The T1R3 activators of the invention may be incorporated into medical and/or dental compositions. Certain compositions used in diagnostic procedures have an unpleasant taste, such as contrast materials and local oral anesthetics. The T1R3 activators of the invention may be used to improve the comfort of subjects undergoing such procedures by improving the taste of compositions. In addition, the T1R3 activators of the invention may be incorporated into pharmaceutical compositions, including tablets and liquids, to improve their flavor and improve patient compliance (particularly where the patient is a child or a non-human animal).

The T1R3 activators of the invention may be comprised in cosmetics to improve their taste features. For example, but not by way of limitation, the T1R3 activators of the invention may be incorporated into face creams and lipsticks. In addition, the T1R3 activators of the invention may be incorporated into compositions that are not traditional foods, beverages, pharmaceuticals, or cosmetics, but which may contact taste membranes. Examples include, but are not limited to, soaps, shampoos, toothpaste, denture adhesive, glue on the surfaces of stamps and envelopes, and toxic compositions used in pest control (e.g., rat or cockroach poison).

### EXAMPLE

### Cloning and Characterization of the T1R3 Gene

The data presented below describes the identification of a novel taste receptor, T1R3, as being Sac. This identification is based on the following observations. T1R3 is the only GPCR present in a 1 million bp region of human genomic DNA centered on the D18346 marker most tightly linked to Sac. Expression of T1R3 is narrowly restricted and is highly expressed in a subset of taste receptor cells. Expression of T1R3 in taste receptor cells overlaps in large part with known and proposed elements of sweet transduction pathways (i.e. α-gustducin, Gγ13. T1R3 is a family 3 GPCR with a large extracellular domain sensitive to proteases (a known property of the sweet receptor). Most tellingly, a polymorphism in T1R3 was identified that differentiated all taster strains of mice from all non-taster strains: T1R3 from non-tasters is predicted to contain an N-terminal glycosylation site that based on modeling of T1R3's structure would be expected to interfere with its dimerization. Hence, not only is T1R3 identified as sac, but based on the model of T1R3 and this polymorphic change it is also likely to be a sweet-responsive (i.e. sweet-liganded) taste receptor.

### Gene Identification

To identify the mouse gene (pseudouridine synthase-like) containing the D18346 marker the D18346 sequence was used as a query sequence in a BlastN screen of the mouse expressed sequence tag (est) database. Each resulting overlapping sequence match was used iteratively to extend the sequence until the nearly full length gene was determined. The resulting contig was translated and the predicted open reading frame was used as a query in a TBlastN search of the High Throughput Genomic Sequence (HTGS) database. This search located a human BAC clone AL139287 containing the human ortholog. Genscan was used to predict genes and exons in this clone. BlastN or TBlastN searches of either the NR or the est databases were used to further define known or unknown genes in this and other clones. Each resulting predicted gene was used in TBlastN or BlastN searches of the HTGS to find overlapping BAC or PAC clones. Each of the overlapping sequences was used in BlastN searches of the HTGS to continue the build of an unordered contig of the region. The predicted genes and exons that resulted from this search were used to partially order over 1 million bases of genomic sequence centered on the pseudouridine synthase-like gene containing the D18346 marker. Two human clones were found to contain T1R3, the aforementioned AL139287 and AC026283. The human T1R3 gene was first predicted by Genscan and subsequently confirmed by RT-PCR of human fungiform taste bud RNA and/or screening of a human taste library. In addition to the above manipulations and searches we used an algorithm (designed to recognize transmembrane spans in genomic sequence) to search all of the human genomic clones on the p arm of human chromosome 1 from 1pter to 1p33 (Sanger Center chromosome 1 mapping project, FC and HW, unpublished). This screen predicted T1R3 as well as T1R1 and T1R2. Human T1R3 lies within 20,000 bp of the D18346 marker and the pseudouridine synthase-like gene and is the only predicted GPCR in this 1 million bp region.

The human predicted gene was then used in a TBlastN screen of the Celera mouse fragment genomic database. Each matching fragment was used to fill gaps and further extend the mouse T1R3 ortholog in repeated BlastN searches. The following mouse fragments were used to build and refine the mouse T1R3 genomic sequence: GA_{—}49588987, GA_{—}72283785, GA_{—}49904613, GA_{—}50376636, GA_{—}74432413, GA_{—}70914196, GA_{—}62197520, GA_{—}77291497, GA_{—}74059038, GA_{—}66556470, GA_{—}70030888, GA_{—}50488116, GA_{—}50689730, GA_{—}72936925, GA_{—}72154490, GA_{—}69808702. Genscan was used to predict the mouse gene from the resulting genomic contig. The predicted mouse T1R3 gene was confirmed by RT-PCR of mouse taste bud RNA. Other genes from the human genomic region centered on D18346 were used to search the Celera mouse fragments database. The sequences from these searches were used to build a mouse genomic contig of this region and confirm the linkage of D18346 with T1R3 in the mouse genome and the micro-synteny of the human and mouse genes in this region. One gap in the genomic sequence, between the 5′-end of T1R3 and the 3′-end of the glycolipid-transferase-like gene was bridged by PCR and confirmed by sequence analysis.

### Northern Hybridization

Total RNAs were isolated from several mouse tissues using the Trizol reagents, then 25 μg of each RNA was electrophoresed per lane on a 1.5% agarose gel containing 6.7% formaldehyde. The samples were transferred and fixed to a nylon membrane by UV irradiation. The blot was prehybridized at 65° C. in 0.25 M sodium phosphate buffer (pH 7.2) containing. 7% SDS and 40 μg/ml herring sperm DNA with agitation for 5 hours; hybridization for 20 hours with the ³²p -radiolabeled mouse T1R3 probe was carried out in the same solution. The membrane was washed twice at 65° C. in 20 mM sodium phosphate buffer (pH 7.2) containing 5% SDS for 40 minutes, twice at 65° C. in the same buffer containing 1% SDS for 40 minutes, and once at 70° C. in 0.1×SSC and 0.1% SDS for 30 minutes. The blot was exposed to X-ray film for 3 days at −80° C. with dual intensifying screens. The ³²p-labeled T1R3 probe was generated by random nonamer priming of a 1.34-kb cDNA fragment of murine T1R3 corresponding to the 5′-end coding sequence using Exo(−) Klenow polymerease in the presence of (α-³²P)-dCTP.

### In Situ Hybridization

33P-labeled RNA probes T1R3 (2.6 kb) and α-gustducin (1 kb)] were used for in situ hybridization of frozen sections (10 μm) of mouse lingual tissue. Hybridization and washing were as described (2). Slides were coated with Kodak NTB-2 nuclear track emulsion and exposed at 4° C. for 3 weeks and then developed and fixed.

### Gene Expression Profiling

Single taste receptor cell RT-PCR products (5 μl) were fractionated by size on a 1.6% agarose gel and transferred onto a nylon membrane. The expression patterns of the isolated cells were determined by Southern hybridization with 3′-end cDNA probes for mouse T1R3, α-gustducin, Gγ13, PLCβ2 and G3PDH. Blots were exposed for five hours at −80° C. Total RNAs from a single circumvallate papilla and a similar-sized piece of non-gustatory epithelium were also isolated, reverse transcribed, amplified and analyzed as for the individual cells.

### Immunocytochemistry

Polyclonal antisera against a hemocyanin-conjugated T1R3 peptide (T1R3-A, aa 829-843) were raised in rabbits. The PLC β2 antibody was obtained from Santa-Cruz Biotechnologies. Ten micron thick frozen sections of human lingual tissue (previously fixed in 4% paraformaldehyde and cryoprotected in 20% sucrose) were blocked in 3% BSA, 0.3% Triton X-100, 2% goat serum and 0.1% Na Azide in PBS for 1 hour at room temperature and then incubated for 8 hours at 4° C. with purified antibody against α-gustducin, or antiserum against T1R3 (1:800). The secondary antibodies were Cy3-conjugated goat-anti-rabbit Ig for T1R3 and fluorescein-conjugated goat-anti-rabbit Ig for PLC β2. PLC β2 and T1R3 immunoreactivities were blocked by preincubation of the antisera with the corresponding synthetic peptides at 10 μM and 20 μM, respectively. Preimmune serum did not show any immunoreactivity. Some sections were double-immunostained with T1R3 and PLC β2 antisera as described (46). Briefly, sections were incubated sequentially with T1R3 antiserum, anti-rabbit-Ig-Cy3 conjugate, normal anti-rabbit-Ig, PLCβ2 antibody and finally with anti-rabbit-Ig-FITC conjugate with intermittent washes between each step. Control sections that were incubated with all of the above except PLCβ2 antibody did not show any fluorescence in the green channel.

### Identification of Sequence Polymorphisms in mT1R3

Based on the sequence of mouse T1R3 obtained from the Celera mouse fragments database, oligonucleotide primers were designed to amplify DNA encoding regions with open reading frames. Total RNA isolated from taste papillae or tail genomic DNA isolated from one taster (C57BL6J) and one non-taster (129/Svev) mouse strain each were used as templates to amplify mouse T1R3 cDNA and genomic DNA using RT-PCR and PCR, respectively. PCR products were sequenced completely in an ABI 310 automated sequencer. Based on the sequence obtained, four sets of oligonucleotide primers were used to amplify the T1R3 regions where polymorphisms were found between the two strains of mice. Genomic DNA from mouse strains DBA/2, BALB/c, C3H/HeJ, SWR and FVB/N, was used as template. The amplicons were purified and directly sequenced. The genealogical tree of these strains of mice was based on Hogan et al, (47) and the Jackson laboratory web site (http://www.jax.org).

### Modeling the Structure of T1R3

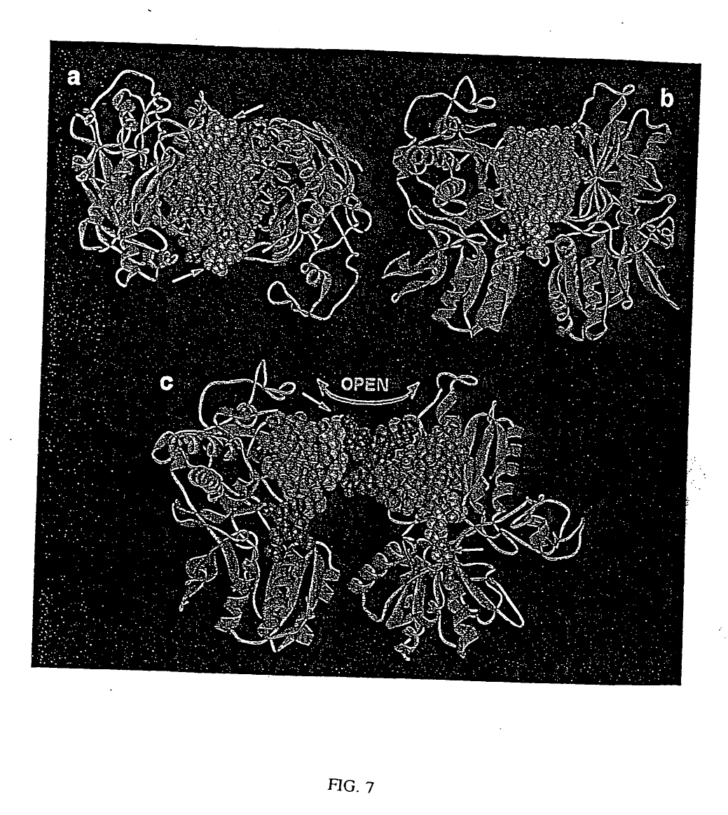

The amino terminal domains (ATDs) of mouse T1R3 and mouse GluR1 were aligned using the ClustalW program (48). The alignment was manually edited to generate an optimal alignment based on structural and functional considerations. Atomic coordinates of the mGluR1 ATD crystal structure (19) were obtained from the protein database and were used along with the alignment as the source of spatial restraints for modeling. The structural model of mouse T1R3 was generated using the program MODELLER (49). The original images for FIG. 7 were created using the programs Insight II and Weblab Viewer (Molecular Simulations Inc.) and then imported into Photoshop where the open view was created and the labels were added.

### Results

### Mapping of the Murine and Human Human Sac Regions

The murine Sac gene is the primary determinant of inter-strain preference responses to sucrose, saccharin, acesulfame, dulcin, glycine and other sweeteners (9-12), however, the molecular nature of the Sac gene product is unknown. Taster vs. non-taster strains of mice display differences in the electrophysiological responses of their taste nerves to sweeteners and sweet amino acids, arguing that Sac exerts its effect on the sweet pathway at the periphery (14, 18). The most likely explanation for these differences is an allelic difference in a gene encoding a sweet-responsive taste transduction element such as a receptor, G protein subunit, effector enzyme or other member of the sweet signaling pathway. It had been speculated that the Sac gene product modified a sweet-responsive receptor (12), was itself a taste receptor (17) or a G protein subunit (14). As a first step toward identifying the nature of the Sac gene we generated a contiguous map of the human genome in this region was generated. Starting with the mouse marker D18346 (16), which maps most closely to the sac locus at 4pter, a novel mouse gene from the est database was identified: D18346 is found in the 3′ untranslated region (UTR) of a novel mouse gene with homology to pseudouridine synthase. At the time this work was initiated the sequence of the human genome was nearly complete (although only partially assembled), while that of mouse was quite incomplete, hence, finished human genomic sequences and unfinished sequences from bacterial artificial chromosome (BAC) and P1 artificial chromosome (PAC) clones known to map to human chromosome 1pter-1p36.33 (syntenic to mouse 4pter) was screened for the ortholog of the novel pseudouridine synthase-like gene containing the D18346 marker. Using the TblastN program the high-throughput human genomic sequence (HTGS) database (NCBI) was searched to identify a PAC clone containing the human ortholog of the pseudouridine synthase-like gene. By repeated Blast searches of the human HTGS with portions of the sequence from this and overlapping PAC and BAC clones we were able to form a contiguous map (“contig”) of 6 overlapping BAC or PAC clones spanning approximately one million bp of human genomic DNA sequence was found.

Using the Genscan gene prediction program we identified the predicted exons and genes within this contig were identified. Twenty three genes were predicted in this region (FIG. 1A), including “pseudouridine synthase-like”, “cleavage and polyadenylation-like”, and “glycolipid transfer-like”; a few genes within this region had been previously identified and/or experimentally verified by others (e.g. disheveled 1, dvl1). The Celera mouse genomic database was searched to identify the murine orthologs of the genes within this region and pieced together the mouse contig (FIG. 1A).

### Identification of a Novel Receptor, T1R3, within the Sac Region

In the screen of the million bp of genomic DNA sequence in the Sac region, only one predicted GPCR gene was found. The gene, which was referred to as T1R3 (for taste receptor one, member three family ), was of special interest because the predicted protein it encodes is most similar to T1R1 and T1R2, two orphan GPCRs expressed in taste cells (17), and because, as will be shown below, it is expressed specifically in taste cells. Human T1R3 (hT1R3) is located about 20 kb from the pseudouridine synthase-like gene, the human ortholog of the mouse gene containing the D18346 marker (FIG. 1A). If T1R3 is Sac, then its proximity to D18346 is consistent with the previously observed very low probability of crossovers between the marker and the Sac locus in F2 crosses and congenic mice (16).

The intron/exon structure of the coding portion of the hT1R3 gene was predicted by Genscan to span 4 kb and contain 7 exons (FIG. 1B). To confirm and refine the inferred amino acid sequence of the predicted hT1R3 protein we cloned and sequenced multiple independent products from polymerase chain reaction (PCR) amplified hT1R3 cDNAs derived from a human taste cDNA library. Based on the nucleotide sequence of the genomic DNA and cDNAs, the hydrophobicity profile and TMpred predictions of membrane spanning regions (FIG. 1C), hT1R3 is predicted to encode a protein of 843 amino acids with seven transmembrane helices and a large 558 amino acid long extracellular domain.

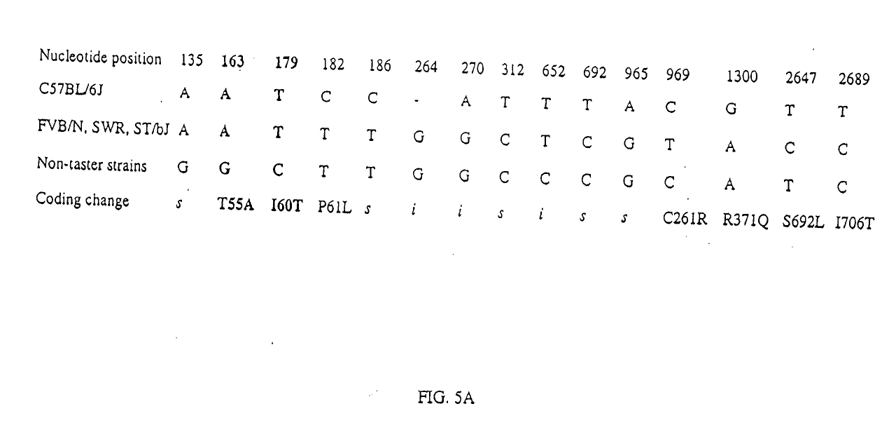

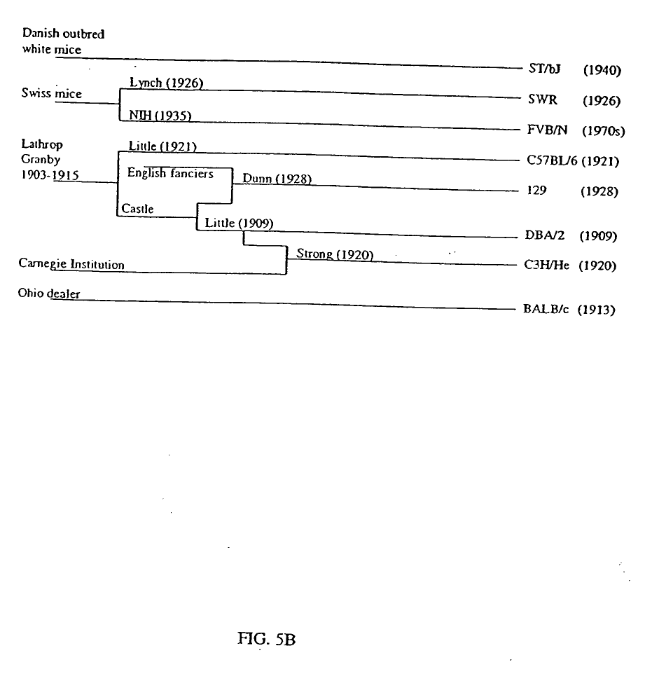

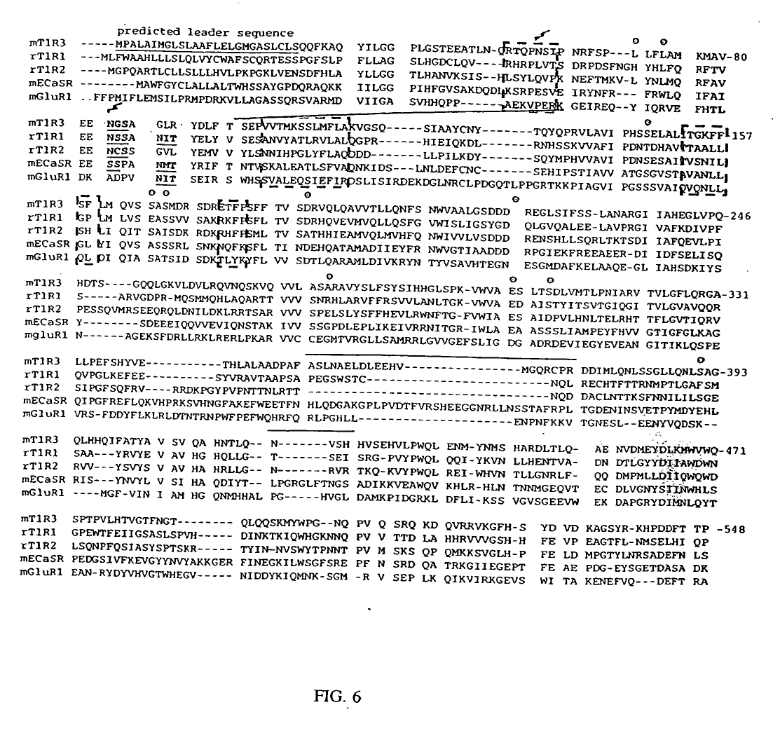

The corresponding mouse T1R3 (mT1R3) genomic sequence was assembled from the Celera mouse genomic fragment database. Several reverse transcriptase (RT)-PCR-generated mouse T1R3 cDNAs derived from taste bud mRNA of different mouse strains were also cloned and sequenced. The coding portion of the mouse T1R3 gene from C57BL/6 spans 4 kb and contains 6 exons; the encoded protein is 858 amino acids long. Polymorphic differences between taster and non-taster strains of mice, and their potential functional significance, are described below (see FIGS. 5 and 6 and related text).

T1R3 is a member of the family 3 subtype of GPCRs, all of which contain large extracellular domains. Other family 3 subtype GPCRs include metabotropic glutamate receptors (mGluR), extracellular calcium sensing receptors (ECaSR), candidate pheromone receptors expressed in the vomeronasal organ (V2R), and two taste receptors, T1R1 and T1R2, of unknown ligand specificity. T1R3 is most closely related to T1R1 and T1R2, sharing ˜30% amino acid sequence identity with each of these orphan taste receptors (T1R1 and T1R2 are ˜40% identical to each other). At the amino acid level hT1R3 is ˜20% identical to mGluRs and ˜23% identical to ECaSRs. The large amino terminal domain (ATD) of family 3 GPCRs has been implicated in ligand binding and dimerization (19). Like other family 3 GPCRs, mT1R3 has an amino-terminal signal sequence, an extensive ATD of 573 amino acids, multiple predicted asparagine-linked glycosylation sites (one of which is highly conserved), and several conserved cysteine residues. Nine of these cysteines are within a region that links the ATD to the portion of the receptor containing the transmembrane domains. The potential relevance of mT1R3's ATD in phenotypic differences between taster and non-taster strains of mice is elaborated below (see FIGS. 5 and 6 and related text).

### Expression of T1R3 mRNA and Protein in Taste Tissue and Taste Buds

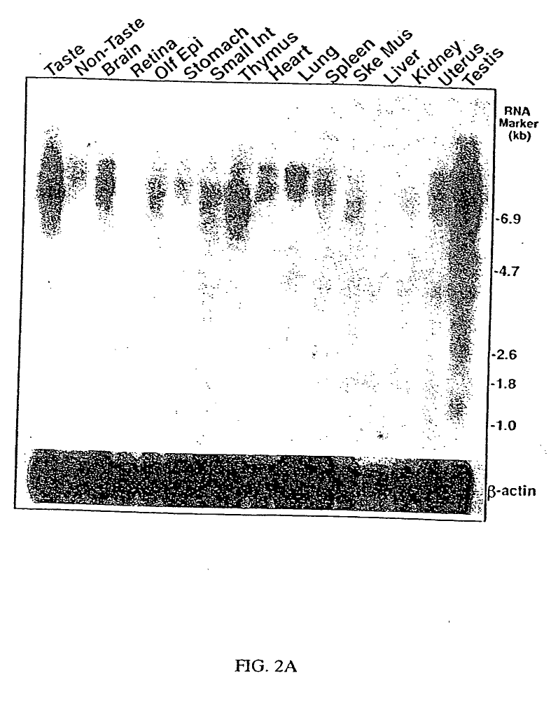

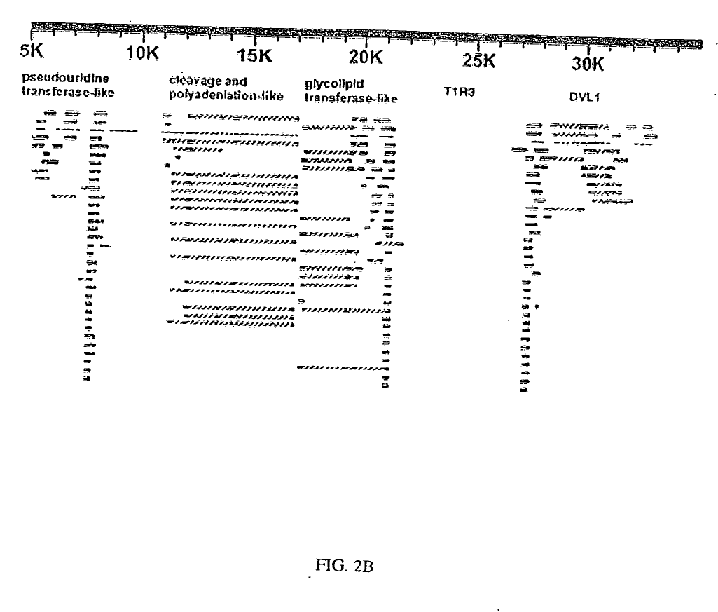

To examine the general distribution of mouse T1R3 in taste and non-taste tissues, northern blot analysis was carried out with a panel of mouse mRNAs. The mouse T1R3 probe hybridized to a 7.2 kb mRNA present in taste tissue, but not expressed in control lingual tissue devoid of taste buds (non-taste) or in any of the several other tissues examined (FIG. 2A). A somewhat larger (˜7.8 kb) mRNA species was expressed at moderate levels in testis, and at very low levels in brain. A smaller (˜6.7 kb) mRNA species was expressed at very low levels in thymus. The 7.2 kb taste-expressed transcript is longer than the isolated cDNAs or Genscan predicted exons, suggesting that additional untranslated sequences may be present in the transcript.

As another measure of the pattern of expression of T1R3 in various tissues the expressed sequence tags (est) database were examined for strong matches to T1R3 and other predicted genes in the Sac region (FIG. 2B). While dvl1, glycolipid transfer-like, cleavage and polyadenylation-like, and pseudouridine synthase-like genes each had numerous highly significant matches to ests from several different tissues, T1R3 showed only a single strong match to an est from colon. This result, consistent with the northern, suggests that expression of T1R3 is highly restricted—such a pattern of under-representation in the est database would fit with T1R3 being a taste receptor.

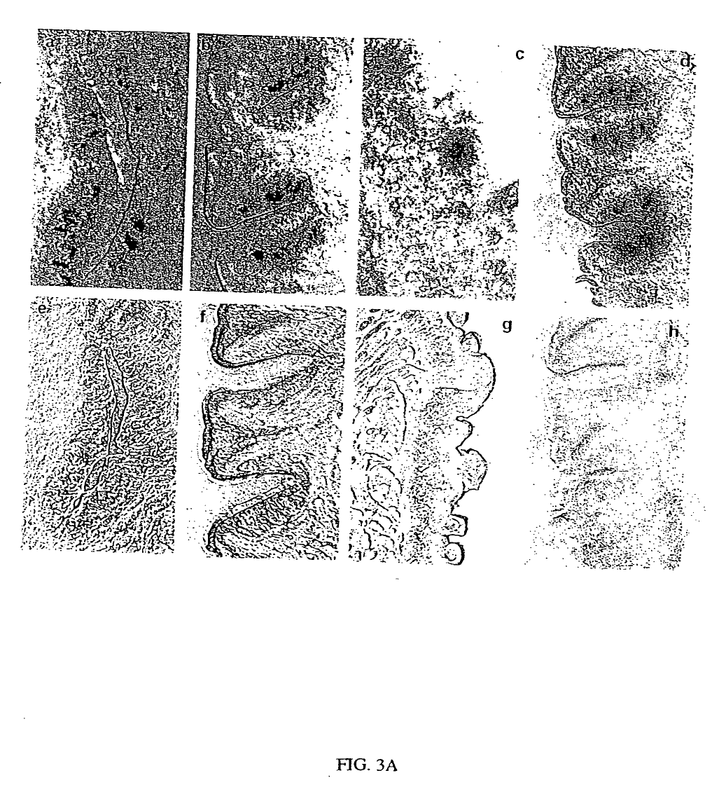

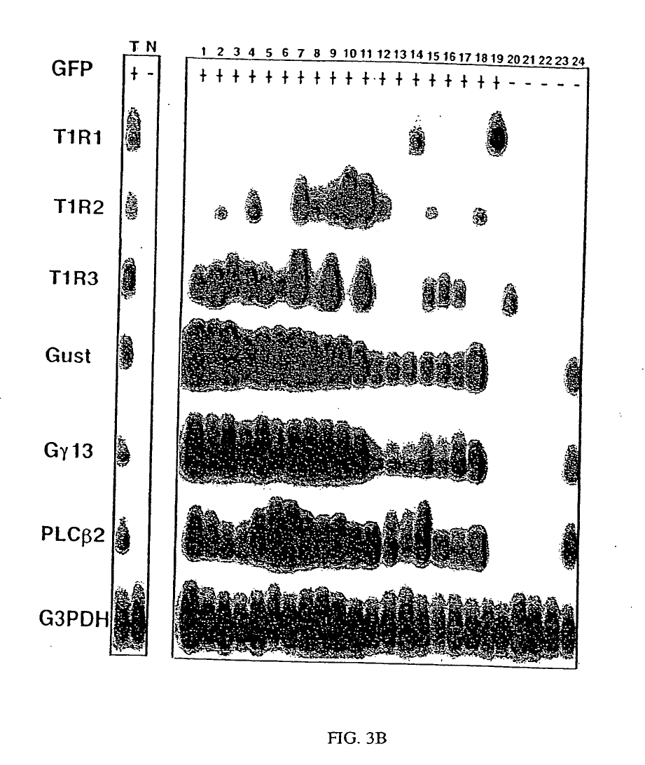

To determine the cellular pattern of T1R3 expression in taste tissue, in situ hybridization was performed: T1R3 was selectively expressed in taste receptor cells, but absent from the surrounding lingual epithelium, muscle or connective tissue (FIG. 3A). Sense probe controls showed no non-specific hybridization to lingual tissue (FIG. 3A). The RNA hybridization signal for T1R3 was even stronger than that for α-gustducin (FIG. 3A), suggesting that T1R3 mRNA is very highly expressed in taste receptor cells. This is in contrast to results with T1R1 and T1R2 mRNAs, which are apparently expressed at lower levels than is α-gustducin (17). Furthermore, T1R3 is highly expressed in taste buds from fungiform, foliate and circumvallate papillae, whereas T1R1 and T1R2 mRNAs each show different regionally variable patterns of expression (T1R1 is preferentially expressed in taste cells of the fungiform papillae and geschmacksstreifen (‘taste stripe’), to a lesser extent in those of the foliate papillae, but rarely in those of the circumvallate papillae; T1R2 is commonly expressed in taste cells of the circumvallate and foliate papillae, but rarely in those of the fungiform papillae or geschmacksstreifen) (17).

To determine if T1R3 mRNA is expressed in particular subsets of taste receptor cells, expression profiling was used (3). First, probes from the 3′regions of mouse clones for T1R3, α-gustducin, Gγ13, PLCβ2 and G3PDH cDNAs were hybridized to RT-PCR-amplified cDNAs from a single circumvallate papilla vs. a similar-sized piece of non-gustatory lingual epithelium. In this way it was determined that mouse T1R3, like α-gustducin, Gγ13 and PLCβ2, was expressed in taste bud-containing tissue, but not in non-gustatory lingual epithelia (FIG. 3B left). The pattern of expression of these genes in individual taste cells was next profiled: the single cell RT-PCR products were hybridized with the same set of probes used above. As previously determined (3), all of the nineteen α-gustducin-positive cells expressed Gβ3 and Gγ13; these nineteen cells also all expressed PLCβ2 (FIG. 3B right). Twelve of these nineteen cells (63%) also expressed T1R3. Only one of the five cells that were α-gustducin/Gβ3/Gγ13/PLCβ2-negative expressed T1R3. From this it was concluded that expression of T1R3 and α-gustducin/Gβ3/Gγ13/PLCβ2, although not fully coincident, overlaps to a great extent. This contrasts with previous in situ hybridization results with taste receptor cells of the foliate papillae in which ˜15% of α-gustducin-positive cells were positive for T1R1 or T1R2 (17).

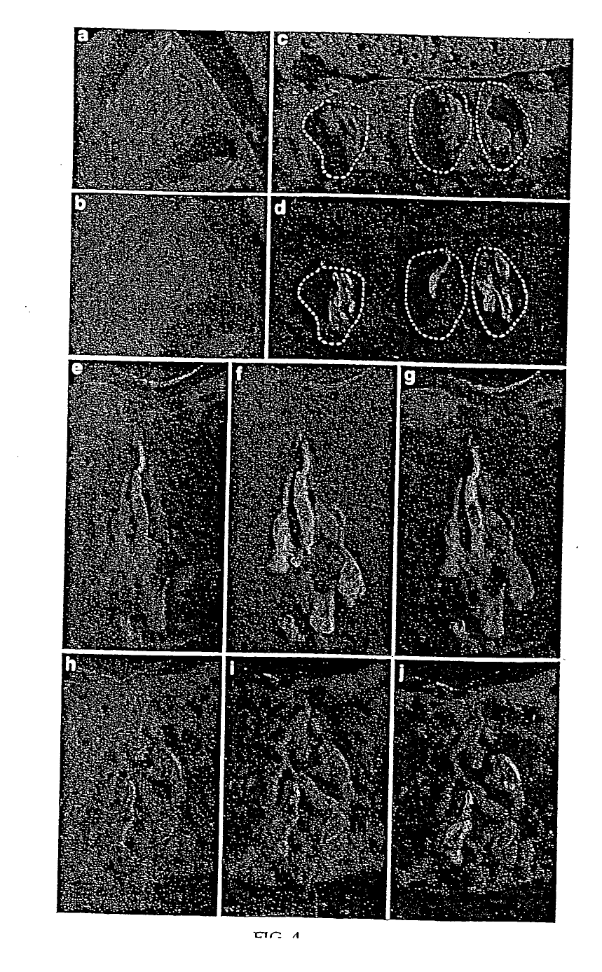

Immunocytochemistry with an anti-hT1R3 antibody demonstrated that about one fifth of taste receptor cells in human circumvallate (FIG. 4AC) and fungiform (FIG. 4EH) papillae were positive for hT1R3. hT1R3 immunoreactivity was blocked by pre-incubation of the hT1R3 antibody with the cognate peptide (FIG. 4B). Longitudinal sections of the hT1R3-postive taste cells displayed an elongated bipolar morphology typical of so called light cells (many of which are α-gustducin-positive), with the immunoreactivity most prominent at or near the taste pore (FIG. 4ACEH). Labeling adjacent sections with antibodies directed against hT1R3 and PLCβ2 showed more cells positive for PLCβ2 than for hT1R3 (FIG. 4CD). Double labeling for hT1R3 and PLCβ2 (FIG. 4EFG), or for hT1R3 and α-gustducin (FIG. 4HIJ) showed many, but not all, cells to be doubly positive (more cells were positive for PLCβ2 or α-gustducin than for hT1R3), consistent with the results from expression profiling. In sum, T1R3 mRNA and protein are selectively expressed in a subset of “-gustducin/PLC$2- positive taste receptor cells as would be expected for a taste receptor.

### A Single Polymorphic Difference in T1R3 may Explain the Sac^{d} Non-Taster Phenotype

C57BL/6 mice carrying the Sac^{b} allele and other so-called taster strains of mice display enhanced preferences and larger chorda tympani nerve responses vs. DBA/2 mice (sac^{d}) and other non-taster strains for several compounds that humans characterize as sweet (e.g. sucrose, saccharin, acesulfame, dulcin and glycine) (10-12, 14, 15, 18). The inferred amino acid sequence of T1R3 from taster and non-taster strains of mice were examined looking for changes that might explain these phenotypic differences (see FIG. 5A). All four non-taster strains (DBA/2, 129/Svev, BALB/c and C3H/HeJ) examined had identical nucleotide sequences despite the fact that their most recent common ancestors date back to the early 1900s or earlier (see FIG. 5B). All four taster strains (C57BL/6J, SWR ,FVB/N and ST/bj) shared four nucleotide differences vs. the non-tasters: nt₁₃₅A→G, nt₁₆₃A→G, nt₁₇₉T→C and nt₆₅₂T→C (the taster nt is listed first). C57BL/6J also had a number of positions at which it differed from all other strains (see FIG. 5A), however, many of these differences were either “silent” alternate codon changes in protein coding regions or substitutions within introns where they would be unlikely to have any pronounced effect. The two coding changes (described as single letter amino acid changes at specific residues; the taster aa is listed first) were T55A and I60T. The I60T change is a particularly intriguing difference as it is predicted to introduce a novel N-linked glycosylation site in the ATD of T1R3 (see below)

To consider the functional relevance of these two amino acid differences in the T1R3 proteins from taster vs. non-taster, the ATD of T1R3 was aligned with those of other members of the type 3 subset of GPCRs (FIG. 6) and the ATD of T1R3 was modeled based on the recently solved structure of the ATD of the related mGluR1 receptor (19) (FIG. 7). The ATD of T1R3 displays 28, 30, 24, and 20% identity to those of T1R1, T1R2, CaSR and mGluR1, respectively (FIG. 6). 55 residues of ˜570 in the ATD were identical among all five receptors. Included among these conserved residues is a predicted N-linked glycosylation site at N85 of T1R3. Based on homology to mGluR1, regions predicted to be involved in dimerization of T1R3 are aa 55-60, 107-118, 152-160, and 178-181 (shown in FIG. 6 within dashed boxes) The I60T taster to non-taster substitution is predicted to introduce a novel N-linked glycosylation site 27 amino acids upstream from the conserved N-linked glycosylation site present in all five 5 receptors. The new N-linked glycosylation site at N58 might interfere with normal glycosylation of the conserved site at N85, alter the structure of the ligand binding domain, interfere with potential dimerization of the receptor, or have some other effect on T1R3 function.

To determine if glycosylation at N58 of the non-taster variant of mT1R3 might be expected to alter the function of the protein we modeled its ATD on that of mGluR1 (19) (FIG. 7). The regions of potential dimerization in T1R3 are very similar to those of mGluR1 and the amino acids in these regions form tight fitting contact surfaces that suggest that dimerization is indeed likely in T1R3. From the model of the three dimensional-structure of the ATD of T1R3 we can see that the novel N-linked glycosylation site at N58 would have a profound effect on T1R3's ability to dimerize (FIG. 7C). The addition of even a short carbohydrate group at N58 (a tri-saccharide moiety has been added in the model in FIG. 7C) would disrupt at least one of the contact surfaces required for stability of the dimer. Therefore, if T1R3, like mGluR1, adopts a dimeric form (either homodimer or heterodimer), then the predicted N-linked glycosyl group at N58 would be expected to preclude T1R3 from forming self-homodimers or heterodimers with any other GPCRs co-expressed with T1R3 using the same dimerization interface. Even if the novel predicted glycosylation site at N58 of non-taster T1R3 is not utilized, the T55A and I60T substitutions at the predicted surface of dimerization may themselves affect the ability of T1R3 to form dimers.

### REFERENCES

1. McLaughlin, S. K. et al., 1992, Nature 357, 563-569

2. Wong, G. T. et al., 1996, Nature 381, 796-800

3. Huang, L. et al., 1999, Nat Neurosci 2, 1055-1062

4. Adler, E. et al., 2000. Cell 10, 693-702

5. Chandrashekar, J. et al., 2000, Cell 100, 703-711

6. Matsunami H et al. Nature 2000, 404:601-604

7. Lindemann, B. 1996, Physiol. Rev. 76, 719-766

8. Gilbertson, T. A. et al., 2000, Curr. Olin. Neurobiol. 10, 519-527

9. Fuller, J. L. 1974, J Hered 65, 33-36.

10. Lush, I. E. 1989 Genet. Res. 53, 95-99

11. Capeless, C. G. and Whitney, G. 1995, Chem Senses 20, 291-298

12. Lush, I. E. et al., 1995, Genet Res 66, 167-174

13. Ninomiya Y. et al. In Chemical Senses Vol. 3, Genetics of Perception and Communication (ed. C. J. Wysocki and M. R. Kare). New York: Marcel Dekker. pp 267-278 (1991).

14. Bachmanov, A. A. et al., 1977, Mammal Genome 8, 545-548

15. Blizzard, D. A. et al, 1999, Chem Senses 24, 373-385

16. Li, X. et al, 2001, Genome 12:13-16

17. Hoon, M. A. et al., 1999, Cell 96, 541-551

18. Frank, M. E. and Blizard, D. A. 1999, Physiol Behav. 67, 287-297

19. Kunishima, N. et al., 2000, Nature 407, 971-977

20. Capretta, P. J., 1970, Psychogenomic Science 21, 133-135

21. Pelz, W. et al., 1973, Physiology and Behavior 10, 263-265

22. Lush, I. E., 1984, Genet. Res. 44, 151-160

23. Lush, I. E., 1986, Genet. Res. 47, 117-123

24. Lush, I. E. and Holland, G., 1988, Genet. Res. 52, 207-212

25. Capeless, C. G. et al., 1992, Behav. Genet. 22, 655-663

26. Kinnamon, S. C. and Margolskee, R. F., 1996, Current Opinion in Neurobiology. 6, 506-513

27. Lum, C. K. L., and Cagan, R. H., 1976, Biochim. Biophys. Acta 421, 380-394.

28. Cagan, R. H., and Morris, R. W., 1979, Proc. Natl. Acad. Sci. USA 76, 1692-1696

29. Farbman, A. I. et al., 1987, Scan. Microsc. 1, 351-357

30. Shimazaki, K. et al.,1986, Biochim. Biophys. Acta 884, 291-298

31. Hiji, T., 1975, Nature 256, 427-429

32. Bernhardt, S. J. et al., J Physiol, 490, 325-336.

33. Nakashima, K., and Ninomiya, Y., 1998, Cell Physiol Biochem 8, 224-230

34. Ming, D. et al., 1998, Proc Natl Acad Sci USA 95, 8933-8938

35. Imoto, T. et al., 1991, Comp. Biochem. Physiol. A Physiol. 100, 309-314

36. Ninomiya Y. et al., 1995, Am. J. Physiol. 268, R1019-R1025

37. Ninomiya Y. et al., 1999, J. Neurophysiol. 81, 3087-3091

38. Rossler, P. et al. 1998, Eur J Cell Biol 77, 253-61

39. Smith, D. V., Margolis, F. L., 1999, Curr Biol 9, R453-455

40. Ninomiya, Y. et al., 1997, Am J Physiol 272, R1002-R1006

41. Yamaguchi, S., 1991, Physiol. Behav. 49, 833-841

42. Chaudhari, N., and Roper, S. D., 1998, Ann. NY Acad. Sci. 855, 398-406

43. Chaudhari, N. et al., 2000, Nat. Neurosci. 3, 113-119

44. Danilova, V. et al., 1999, Sus scrofa. Chem Senses 24, 301-316

45. Ninomiya, Y. et al., 2000, J Nutr. 130, 950S-953S

46. Bakre, M. M. et al., 2001, Submitted (2001).

47. Hogan, B., Beddington, R., Costantini, F. & Lacy, E. Manipulating the mouse embryo: a laboratory manual, (Cold Spring Harbor Laboratory, Cold Spring Harbor, 1994).

48. Thompson, J. D. et al., Nucleic Acids Res. 22, pp. 4673-4680.

49. Sali, A. and Blundell, T. L., 1993, J Mol. Biol 234, 779-815.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Various references are cited herein, the disclosures of which are incorporated by reference in their entireties.

We claim:

1. An isolated nucleic acid molecule comprising a nucleotide sequence that encodes the amino acid sequence shown in FIG. 1B.

2. The isolated nucleic acid molecule of claim 1 comprising the DNA sequence of FIG. 1B.

3. The isolated nucleic acid molecule of claim 2 comprising a nucleotide sequence that encodes the amino acid sequence shown in FIG. 1B.

4. An isolated nucleic acid molecule comprising a nucleotide sequence that hybridizes to the nucleotide sequence of claim 1 or 2 under stringent conditions and encodes a functionally equivalent gene product.

5. An isolated nucleic acid molecule comprising a nucleotide sequence that hybridizes to the nucleic acid of claim 1 or 2 under moderately stringent conditions and encodes a functionally equivalent T1R3 gene product.

6. An isolated nucleic acid molecule that is a T1R3 antisense molecule.

7. An isolated polypeptide comprising the amino acid sequence of FIG. 1B.

8. An isolated polypeptide comprising the amino acid sequence encoded by a nucleotide sequence that hybridizes to the nucleotide sequence of claim 1 or 2 under stringent conditions and encodes a functionally equivalent gene product.

9. An isolated polypeptide comprising the amino acid sequence encoded by a nucleotide sequence that hybridizes to the nucleotide sequence of claim 1 or 2 under moderately stringent conditions and encodes a functionally equivalent gene product.

10. A purified fragment of a T1R3 protein comprising a domain of the T1R3 protein selected from the group consisting of the amino terminal domain, transmembrane domain and cytoplasmic domain.

11. A chimeric protein comprising a fragment of a T1R3 protein consisting of at least 6 amino acids fused via a covalent bond to an amino acid sequence of a second protein, in which the second protein is not a T1R3 protein.

13. An antibody which is capable of binding a T1R3 protein. A recombinant cell containing the nucleic acid of claim 4 or 5.

14. A method of producing a T1R3 protein comprising growing a recombinant cell containing the nucleic acid of claim 4 or 5 such that the encoded T1R3 protein is expressed by the cell, and recovering the expressed T1R3 protein.

15. A method for identifying a compound that induces the perception of a sweet taste comprising:
(i) contacting a cell expressing the T1R3 channel protein with a test compound and measuring the level of T1R3 activation;
(ii) in a separate experiment, contacting a cell expressing the T1R3 receptor protein with a vehicle control and measuring the level of T1R3 activation where the conditions are essentially the same as in part (i); and
(iii) comparing the level of activation of T1R3 measured in part (i) with the level of activation of T1R3 in part (ii), wherein an increased level of activated T1R3 in the presence of the test compound indicates that the test compound is a T1R3 inducer.

16. A method for identifying a compound that inhibits the perception of a sweet taste and/or promotes the perception of a sweet taste comprising:
(i) contacting a cell expressing the T1R3 receptor protein with a test compound in the presence of a sweet tastant and measuring the level of T1R3 activation;
(ii) in a separate experiment, contacting a cell expressing the T1R3 receptor protein with a sweet tastant and measuring the level of T1R3 activation, where the conditions are essentially the same as in part (i); and
(iii) comparing the level of activation of T1R3 measured in part (i) with the level of activation of T1R3 in part (ii), wherein a decrease level of activation of T1R3 in the presence of the test compound indicates that the test compound is a T1R3 inhibitor.

17. A method for identifying an inhibitor of sweet taste in vivo comprising:
(i) offering a test animal the choice of consuming either (a) a composition comprising a sweet tastant or (b) the composition comprising the sweet tastant as well as a test inhibitor; and
(ii) comparing the amount of consumption of the composition according to (a) or (b), wherein greater consumption of the composition according to (a) has a positive correlation with an ability of the test inhibitor to inhibit the perception of sweet taste associated with the tastant.

18. A method for identifying an activator of sweet taste in vivo comprising:
(i) offering a test animal the choice of consuming either (a) a control composition or (b) the composition comprising a test activator; and
(ii) comparing the amount of consumption of the composition according to (a) or (b), wherein greater consumption of the composition according to (b) has a positive correlation with an ability of the test activator to activate the perception of sweet taste.

19. A method of inhibiting a sweet taste resulting from contacting a taste tissue of a subject with a sweet tastant, comprising administering to the subject an effective amount of a T1R3 inhibitor.

20. A method of producing the perception of a sweet taste by a subject, comprising administering, to the subject, a composition comprising a compound that acts as an activator of T1R3.

21. A method of producing the perception of a sweet taste by a subject, comprising administering, to the subject, a composition comprising a compound that acts as a sweetness activator.
